(19) United States
(12) Patent Application Publication
(10) Pub. No.: US 2014/0220154 A1
(43) Pub. Date: Aug. 7, 2014

(54) METHOD OF TREATING PATHOLOGIC HETEROTOPIC OSSIFICATION

(71) Applicants: Jean Regard, Silver Spring, MD (US); Yingzi Yang, Bethesda, MD (US)

(72) Inventors: Jean Regard, Silver Spring, MD (US); Yingzi Yang, Bethesda, MD (US)

(73) Assignee: The United States of America, as represented by the Secretary, Dept. of Health and Human Service, Bethesda, MD (US)

(21) Appl. No.: 14/130,220

(22) PCT Filed: Jun. 28, 2012
(86) PCT No.: PCT/US2012/044613

§ 371 Date: Apr. 2, 2014

Related U.S. Application Data
(60) Provisional application No. 61/504,041, filed on Jul. 1, 2011.

(51) Int. Cl. A61K 33/36; A61K 31/506; A61K 31/444

(52) U.S. Cl. CPC A61K 33/36; A61K 31/444; A61K 31/506
USPC 424/623; 514/333; 514/256; 435/375

(57) ABSTRACT

What is described is a method of preventing or treating heterotopic ossification, vascular calcification, and pathologic mineralization, comprising administering an drug, wherein the drug is an antagonist of the Hedgehog (Hh) pathway. For example the antagonist consists of arsenic trioxide, sodium arsenite, phenylarsine, GANT-58, GANT-61, zerumbone vismodegib (GDC-0449, Genentech), bevacizumab, gemcitabine, nab-paclitaxel, FOLFIR, FOLFOX, RO4929097, cixutumumab, cisplatin, etoposide, LDAC, decitabine, daunorubicin, cytarabin, rosiglitazone, goserelin, leuprolide, capecitabine, fluorouracil, leucovorin, oxaliplatin, irinotecan, diclofenac, BMS-833923 (XL139), IPI-926—Infinity Pharmaceuticals, Inc., LDE225, LEQ506—Novartis Pharmaceuticals, TAK-441 Millennium Pharmaceuticals, Inc., and PF-04449913—Pfizer, alone or in combination therapy. The method targets pluripotent mesenchymal cells, wherein the antagonist inhibits expression of a gene encoding a Hh pathway component, for example by decreasing levels of mRNA encoded Ptch1, Gli1 or HIP.

### CROSS REFERENCE TO RELATED APPLICATION

This applications claims the benefit of U.S. Provisional Application No. 61/504,041 filed Jul. 1, 2011, the contents of which are hereby incorporated by reference in its entirety.

### TECHNICAL FIELD

What is described are methods of using antagonists of the Hedgehog pathway to treat heterotopic ossification, vascular calcification, or pathologic mineralization, and to prepare medicaments for treating these diseases.

### BACKGROUND

Heterotopic ossification (HO) can result from osteoid formation of mature lamellar bone in soft tissue sites outside the skeletal periosteum (skeletal system). HO most commonly occurs around proximal limb joints. This osteoid formation often is associated with an inflammatory phase characterized by local swelling, pain, erythema and sometimes fever. This pathological process may occur in sites such as the skin, subcutaneous tissue, skeletal muscle, and fibrous tissue adjacent to joints. Bone may also form in walls of blood vessels as well as in ligaments. Lesions range from small clinically insignificant foci to massive deposits throughout the body.

HO presents rarely as a hereditary disorder, and is sometimes associated with lower motor neuron disorders. More commonly it is associated with spinal cord injury, trauma and brain injuries, burns, fractures, muscle contusion, and joint arthroplasty. HO is a severe complication of hip surgery, acetabular and elbow fracture surgery. It may occur in patients who are on neuromuscular blockade to manage adult respiratory distress syndrome, and in patients with nontraumatic myelopathies. Following combat-related trauma, for example amputation, HO is a frequent occurrence and a common problem. HO may result in joint contracture and ankylosis, pain, spasticity, swelling fever, neurovascular compression, pressure ulcers, and significant disability.

HO can also be caused by genetic diseases such as progressive osseous heteroplasia (POH; MIM #166350) and Fibrodysplasia Ossificans Progressiva (FOP; MIM #135100). POH is associated with inactivating mutation in the GNAS gene, which encodes Gαₛ, the alpha subunit of the stimulatory guanine nucleotide binding protein that acts downstream of many G protein-coupled receptors in activating adenylyl cyclase (Kaplan, et al. 1994, J Bone Joint Surg Am 76, 425-436; Shore, et al., 2002, N Engl J Med 346, 99-106; and Eddy, et al., 2000, J Bone Miner Res 15, 2074-2083). Patients with inactivating mutations in GNAS can also suffer from Albright's hereditary osteodystrophy (AHO) when the genetic mutations are inherited from the mother. Clinically, POH presents during infancy with dermal and subcutaneous ossifications that progress during childhood into skeletal muscle and deep connective tissues (e.g. tendon, ligaments, fascia). Over time these ossifications lead to joint stiffness, bone and joint fusions and growth retardation of the affected limbs. Currently, patients with POH undergo aggressive surgical resection of ectopic bone to abrogate spreading of the lesion. This often results in partial or full amputation of limbs and lesions frequently return (Kaplan, et al., 2000, J Bone Miner Res 15, 2084-94; and Shehab, et al., 2003, J Nucl Med 43, 346-353), which underscores the importance of developing improved therapeutic interventions. Observations made in patients with POH suggest that mesenchymal stem cells present in soft tissues inappropriately differentiate into osteoblasts and begin to deposit bone. Due to a lack of both in vitro and in vivo animal models, the pathogenesis of POH remains unknown and, like all forms of HO, lacks adequate treatments.

During HO there must be an inciting event, usually an episode of trauma which may result in hematoma. There is usually a signal from the site of injury, suggested to be bone morphogenetic protein(s). For HO progression, there must be a supply of pluripotent (multipotent) mesenchymal cells, which can differentiate into osteoblasts or chondroblasts, and an environment conducive to the continued production of heterotopic bone. A mouse model of FOP expressing a strong constitutively active ALK2 R206H mutant, was found to be useful in identifying a selective agonist to nuclear retinoic acid receptor-α (RAR-α) in mesenchymal cells. RAR-α agonists were found to partly inhibit HO, while an agonist to RAR-γ was found to be a potent inhibitor of intramuscular and subcutaneous HOin FOP models (Shimono et al., 2011, Nature Medicine 17:454-60).

HO in FOP occurs through endochondral ossification mechanism where cartilage formation precedes osteoblast differentiation whereas HO in POH occurs through intramembranous ossification where osteoblasts differentiate directly from mechenchymal progenitor cells. Non-genetic forms of HO occur through mechanisms of both endochondral and intramembranous ossification. Therefore, understanding the molecular and cellular mechanisms underlying POH will contribute significantly to our understanding of HO, which is essential in finding methods for treating HO, a pressing need that can only be met by a therapeutic that targets potential signalling pathways associated with this disease.

### SUMMARY

One aspect of the invention is a method of preventing or treating HO comprising administering a drug, wherein the drug is an antagonist of the Hedgehog pathway. The antagonist is selected from the group consisting of zerumbone epoxide, staurosporinone, 6-hydroxystaurosporinone, arcyriaflavin C, 5,6-dihyroxyarcyriaflavin A, physalin F, physalin B, cyclopamine, HPI-1, HPI-2, HPI-3, and HPI-4, arsenic trioxide (ATO), sodium arsenite, phenylarsine, GANT-58, GANT-61, and zerumbone. Preferably the antagonist is an arsenic compound, most preferably ATO. The antagonist may be administered by injection, preferably by an infusion. If the antagonist is ATO, the preferred dosage ranges between 0.05 to 0.20 mg/kg/day. The antagonist may be administered orally. An embodiment of the invention is a method whereby the antagonist targets pluripotent mesenchymal cells, preferably to prevent proliferation or differentiation of the mesenchymal cells.

Another embodiment of the invention is the method of treating HO in which the antagonist alters expression of a gene expressed in the mesenchymal cells, preferably a gene that encodes a component of the Hedgehog pathway. These genes may be selected from a gene family consisting of Hh, PTCH, GLI, and SMO, preferably a gene is selected from the group consisting of Shh, Dhh, Ihh, Ptch1, Ptch2, Gli1, Gli2, Gli3, and Smo. Preferably, the antagonist inhibits expression of the gene, including decreasing levels of mRNA encoded by the gene, particularly Ptch1, Gli1 or HIP.

Another aspect of the invention is a method of inhibiting formation of heterotopic ossification comprising administering an antagonist of the Hedgehog pathway, particularly to a subject that is susceptible to HO. The subject is preferably a mammal, most preferably a human patient. Such patients include those who experienced trauma, including spinal cord injury, trauma and brain injuries, burns, fractures, muscle contusion, joint arthroplasty, lower motor neurone disorders, hereditary disorders, or combat-related trauma. HO amenable to treatment may be diagnosed by computed tomography, bone scintigraphy, ultrasonography, or X-radiography.

Another aspect of the invention is use of an antagonist of the hedgehog pathway for preparing a medicament for treating HO, vascular calcification, or pathologic mineralization. The antagonist is selected from the group consisting of zerumbone epoxide, staurosporinone, 6-hydroxystaurosporinone, arcyriaflavin C, 5,6-dihyroxyarcyriaflavin A, physalin F, physalin B, cyclopamine, HPI-1, HPI-2, HPI-3, and HPI-4, ATO, sodium arsenite, phenylarsine, GANT-58, GANT-61, and zerumbone.

Another aspect of the invention is a method of using an antagonist of the Hedgehog pathway, comprising administering said antagonist to a subject in need thereof to prevent or treat HO, vascular calcification, or pathologic mineralization. The amount of said antagonist administered to the subject are sufficient to reduce levels of HO in the subject.

Another aspect of the invention is a method of using an antagonist of the Hedgehog pathway, comprising exposing mesenchymal cells to said antagonist to prevent activation of said cells. In an embodiment of this method the antagonist prevents proliferation or differentiation of mesenchymal cells. In another embodiment, mesenchymal cell activation results in increased expression of a gene encoding a component of the Hedgehog pathway. The gene is selected from a gene family consisting of Hh, PTCH, GLI, and SMO, specifically, a gene is selected from the group consisting of Shh, Dhh, Ihh, Ptch1, Ptch2, Gli1, Gli2, Gli3, and Smo. In another embodiment, the antagonist inhibits expression of the gene, preferably by decreasing levels of mRNA encoded by the gene, most preferably wherein the gene is Ptch1, Gli1 or HIP.

### BRIEF DESCRIPTION OF THE DRAWINGS

FIG. 1: HO formation in Prx1-cre+; Gαₛ^{flox/-} mice and not in littermate control mice. Alizarin Red stains bone and Alcian Blue stains cartilage. (A) A control forelimb at postnatal day 4 shows normal limb architecture. (B) A mutant limb at P4 with profound HO (arrows) forming in soft tissues. (C) A control hind limb at P18 shows normal limb architecture. (D) A mutant limb at P18 with profound HO (arrows) and ossification of the Achilles tendon (arrowhead).

FIG. 2: HO in Prx1-cre+; Gαₛ^{flox/-} mice. (A) Von Kossa staining demonstrates the presence of mineralized tissue in subcutaneous spaces in the limb of Prx1-cre+; Gαₛ^{flox/-} mice (arrow). A nuclear fast red counterstain allows the visualization of tissue architecture. (B) Immunohistochemistry for the early osteoblastic marker osterix demonstrates its presence (brown nuclear staining; arrows) in cells surrounding a subcutaneous ossicle (light blue, Alcian Blue staining). A nuclear fast red counterstain allows the visualization of tissue architecture.

FIG. 3: Mineralization is seen around small and large blood vessels in Prx1-cre+; Gαₛ^{flox/-} and Dermo1-cre+; Gαₛ^{flox/-} mice. (A) Von Kossa staining shows the association of mineralized tissue with blood vessels in the limb of Prx1-cre+; Gαₛ^{flox/-} mice (arrows). A nuclear fast red counterstain allows the visualization of tissue architecture. (B) An Alizarin Red-Alcian Blue stain of the heart, great vessels and upper airway of a littermate control and a Dermo1-cre+; Gαₛ^{flox/-} mouse. Mineralization is observable around the large vessels (arrows) of Dermo1-cre+; Gαₛ^{flox/-} mice, but not in control mice.

FIG. 4: Elevated expression of Hh pathway markers in Prx1-cre+; Gαₛ^{flox/-} mice. (A) qRT-PCR was performed on RNA isolated from E14.5 forelimbs of either littermate control or Prx1-cre+; Gαₛ^{flox/-} mice. As expected, RNA for Gαₛ is reduced Importantly, RNA for three markers of Hh pathway activation (Ptch1, Gli1, HIP) are elevated in mutant mice relative to control mice (mean±SD; n=4; *p<0.05). (B) In situ hybridization was performed on E14.5 forelimbs collected from littermate control and Prx1-cre+; Gαₛ^{flox/-} mice using a Ptch1 antisense probe. Elevated expression of Ptch1 is observable in the mutant limb, including in areas where HO is first seen. (C) In situ hybridization was performed on E14.5 forelimbs collected from littermate control and Prx1-cre+; Gαₛ^{flox/-} mice using a Gli1 antisense probe. Elevated expression of Gli1 is observable in the mutant limb, including in areas where HO is first seen. (D) In situ hybridization was performed on E14.5 forelimbs collected from littermate control and Prx1-cre+; Gαₛ^{flox/-} mice using a HIP antisense probe. Elevated expression of HIP is observable in the mutant limb, including in areas where HO is first seen.

FIG. 5: Accumulation of full length Gli3 in Prx1-cre+; Gαₛ^{flox/-} mouse limbs. Tissue was isolated from E18.5 limb and western blotting was performed to query for the presence of full length Gli3. Blotting for tubulin demonstrates equal loading of protein in each lane. Blotting for Gαₛ demonstrates reduced levels in mutant lanes relative to control. Blotting for Gli3 demonstrates a decrease in the repressor form (Gli1-R) and increase in the full length form (Gli1-FL), consistent with elevated Hh signaling in mutant mice.

FIG. 6: Gαₛ and Ptch1 interact genetically to produce Hh phenotypes. Male Gαₛ^{flox/-};Ptch1^{flox/-} mice were mated to Gαₛ^{flox/flox} female mice to look for evidence of a genetic interaction between Gαₛ and the Hh pathway. Dermo1-cre+; Gαₛ^{flox/-};Ptch1^{1+/+} and Dermo1-cre+; Gαₛ^{flox/-};Ptch1^{flox/-} mice appear grossly normal at E13.5. Dermo1-cre+; Gαₛ^{flox/-};Ptch1^{flox/+} mice are severely affected with dramatic skeletal defects demonstrating an interaction between Gαₛ and the Hh pathway in the development of skeletal tissues.

FIG. 7: Removal of Gαₛ from bone marrow mesenchymal cells (BMMC) increases Hh signaling and promotes osteoblastic differentiation. BMMCs were isolated from Gαₛ^{flox/flox} mice and infected with a Cre-containing adenovirus to remove Gαₛ. Cells were grown to confluence and place in osteogenic media for 7 days. (A) qRT-PCR analysis demonstrates an increase in Hh pathway signaling at confluence following removal of Gαₛ. The increased expression of osteoblast markers (mean±SD; n=3; *p<0.05) is evidence of an associated increase in osteoblast differentiation. (B) Accelerated osteoblast differentiation is also demonstrated by increased Alizarin red staining.

FIG. 8: ATO inhibits elevated Hh signaling in Prx1-cre+; Gαₛ^{flox/-} mouse limbs. Control and Prx1-cre+; Gαₛ^{flox/-} mouse limbs were isolated at E14.5 and cultured in vitro for 4 days in the BGJB media containing vehicle or 5 μM ATO. Limbs were then collected, RNA isolated and qRT-PCR was performed for markers of Hh pathway activation (Ptch1, Gli1, HIP). In the absence of ATO, expression of these markers was elevated relative to control (6-fold, 5-fold and 17-fold, respectively). In the presence of 5 μM ATO expression of these markers was significantly reduced (mean±SD; n=3; *p<0.05).

FIG. 9: A schematic showing the method of administration and tissue collection to study HO development in adult mice with Adenovirus cre recombinase (Ad-Cre)-driven loss of Gαₛ. Ad-Cre injections (100 μL of 1:10 diluted virus in PBC) where administered subcutaneously above Gα_{sflox/flox} mouse right limbs, while the control Adenovirus GFP (Ad-GFP) subcutaneous injections were given subcutaneously over Gα_{sflox/flox} mouse left limbs when these mice were 1 months old. 6 weeks following the adenovirus injection, the mice were sacrificed and limbs (with intact subcutaneous tissue) were collected for skeletal preparations and histological evaluations.

FIG. 10: HO formation with loss of Gαₛ in adult mice. Alizarin Red stains bone and Alcian Blue stains cartilage. (A) A control forelimb from a 10 week old Gα_{sflox/flox} mouse, 6 weeks after Ad-GFP administration, shows normal limb architecture. (B) A mutant limb from a 10 weeks old Gα_{sflox/flox} mouse, 6 weeks after Ad-Cre administration with profound HO (arrowhead) forming in soft tissues. (C) A control hind limb from a 10 weeks old Gα_{sflox/flox} mouse, 6 weeks after Ad-GFP administration, shows normal limb architecture. (D) A mutant limb from a 10 weeks old Gα_{sflox/flox} mouse, 6 weeks after Ad-Cre administration with profound HO (arrowhead) forming in soft tissues over the endogenous bone.

FIG. 11: HO in adult loss of Gαₛ in Gα_{sflox/flox} mice. (A) H and E staining suggests the presence of mineralization in subcutaneous tissue about right limbs Gα_{sflox/flox} mice (arrow), which received Ad-Cre injection, while no subcutaneous mineralization observed on the control left side in Gα_{sflox/flox} mouse left limb, following Ad-GFP injection. A nuclear fast red counterstain allows the visualization of tissue architecture. (B) Von Kossa staining demonstrates the presence of mineralized tissue in subcutaneous tissue above right limbs Gα_{sflox/flox} mice (arrow), which received Ad-Cre injection, while no subcutaneous mineralization observed on the control left side in Gα_{sflox/flox} mouse left limb, following Ad-GFP injection. A nuclear fast red counterstain allows the visualization of tissue architecture. (C) Immunohistochemistry for the early osteoblastic marker osterix demonstrates its presence (brown nuclear staining; arrows) in cells in the subcutaneous ossicle. A nuclear fast red counterstain allows the visualization of tissue architecture.

FIG. 12: ATO inhibits HO formation in Prx1-cre+; Gαₛ^{flox/-} mice. Pregnant females were injected with either 5 μg/kg ATO or vehicle at E13.5, E15.5 and E17.5 and embryos were collected at E18.5. An Alizarin Red-Alcian Blue skeletal prep was performed to assay for the presence of HO between the digits. Both forelimbs and hind limbs from Prx1-cre+; Gαₛ^{flox/-} mouse pups isolated from female mice receiving ATO injections contained reduced levels of HO relative to Prx1-cre+; Gαₛ^{flox/-} mouse pups isolated from female mice receiving vehicle injections (arrows).

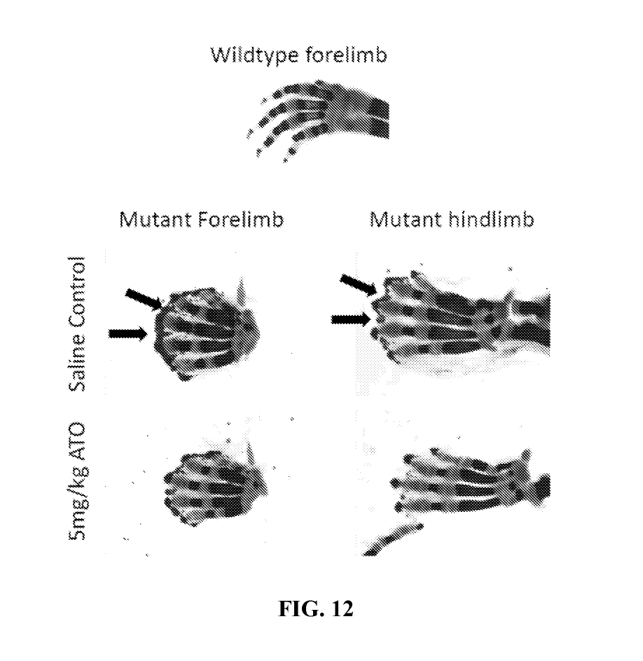

FIG. 13: Removal of Gαₛ from bone marrow mesenchymal cells (BMMC) promotes osteoblastic differentiation, which is inhibited by Hh antagonist, GANT-58 treatment in a dose-dependent manner. BMMCs were isolated from Gα_{sflox/flox} mice and infected with a Cre-containing adenovirus (ad-Cre) to remove Gαₛ or GFP-containing adenovirus (Ad-GFP) control. Cells were grown to confluence and placed in osteogenic media for 7 days. Von Kossa staining analysis demonstrates an increase in tissue mineralization at confluence following removal of Gαₛ (panel b) as compared to Ad-GFP treatment (panel a), which is inhibited by GANT-58 in a dose-dependent manner (panels d and f compared to panel b). No effect of GANT-58 observed in Ad-GFP treated cells (panels c and e compared to a). Similar inhibition of osteoblast differentiation was observed by Alizarin red staining (panels j and l compared to h).

FIG. 14: HO in with adult gain of function of hedgehog effector protein Smoothed, in Gt(ROSA)26Sortm1(Smo/YFP)Amc/J mice following Ad-Cre subcutaneous injection. Ectopic bone forms over the right forelimb and hind limb following Ad-Cre-mediated loss of Gαₛ in Gα_{sflox/flox} mice and not on the control side left limb with Ad-GFP injection. Alizarin Red stains bone and Alcian Blue stains cartilage. (A) A control forelimb from a 10 weeks old Gt(ROSA)26Sortm1(Smo/YFP)Amc/J mouse, 8 weeks after Ad-GFP administration, shows normal limb architecture. (B) A mutant limb from a 10 weeks old Gt(ROSA)26Sortm1(Smo/YFP)Amc/J mouse, 8 weeks after Ad-Cre administration with profound HO (arrowhead) forming in soft tissues.

### DETAILED DESCRIPTION OF ILLUSTRATIVE EMBODIMENTS

What is described in a method of using antagonists of the Hedgehog pathway to treat HO, vascular calcification, and/or pathologic mineralization.

The term “heterotopic ossification” refers to the frequent sequela of central nervous system injury. It is encountered in certain embodiments, in cases of spinal cord injury, head injury, cerebrovascular accident and burns. In one embodiment, neurogenic heterotopic ossification is not associated with local trauma. Osseous trauma is associated with an increased incidence of heterotopic ossification distal to the trauma site, or due to the extent of the original cerebral injury in other embodiment. In one embodiment, the onset of heterotopic ossification may be as early as two weeks postinjury and patients remain susceptible to its onset through the first nine months after injury. In one embodiment, alkaline phosphatase level is raised in the presence of calcium deposition, with the development of heterotopic ossification preceding the elevation of serum alkaline phosphatase. In one embodiment, the hip appears to be the most common site of heterotopic ossification formation, occurring with almost equal frequency in the upper extremities and at both the elbow and the shoulder from craniocerebral injury.

Genetic diseases fibrodysplasia ossificans progressive (FOP) and progressive osseous heterplasia (POH) the most severe manifestations of heterotopic bone formation. FOP occurs rarely and is a result of a mutation in ACVR1, which encodes a bone morphogenetic protein type I receptor. Patients with POH have inactivating mutations of the GNAS gene, which also can give rise to Albright's hereditary osteodystrophy (AHO) when the mutations are inherited from the mother.

Myositis ossifican circumscripta is characterized by the intramuscular proliferation of fibroblasts, new bone, and/or cartilage.

The earliest opportunity for acute treatment, HO can be reliably diagnosed by computed tomography, bone scintigraphy and ultrasonography. Two to six weeks later plain radiography can detect it. Bony maturation occurs within six months.

Conventional treatment usually involves non-steroidal anti-inflammatory drugs (indomethecin, rofecoxib), or bisphosphonate (etidronate, pamidronate), Coumadin/warfarin, salicylates, and/or local radiation are administered. Often, surgery is the only option for treatment.

Outcome of treatment can be measured by standard radiological grading system for HO, changes in range of motion in the affected joint measured by goniometry, mean length of time to objective improvement of HO-related clinical symptoms or signs, changes in standardized functional or joint-specific measures

What is meant by “vascular calcification,” or equivalently vascular ossification calcification (VOC), and “pathologic mineralization” is the result of deposition of calcium salts in the neointima of atheromatous plaques or in the media of vascular beds. Vascular smooth muscle cells (VSMCs) become multipotent mesenchymal cells that can transform into osteocytic- or chondrocytic-like cells. VOC and pathological mineralization are major risk factors for cardiovascular morbidity and mortality. Clinically there is a need to develop therapies to prevent calcification in situations of atherosclerosis, chronic kidney disease, type II diabetes, especially in hemodialysis patients. A chronic inflammatory state is commonly associated with VOC in these situations. VOC can usually be detected by X-ray or CT.

Apoptosis and vesicle release from VSMCs are crucial initiating events in VOC. Inflammatory cytokines may play an initiating role. Following the initiating even, a number of secondary responses can give rise to VOC, including hypercalcemia, hyperphosphatemia, oxidative stress, and aging. BMP, osteoprotegerin (OPG) and other osteogenic signalling pathways are also implicated in development of VOC, just as these pathways are central in HO. Evidence of OPG-mediated receptor activation of RANK and RANKL have been implicated in VOC. VOC is treated in a variety of ways. Antihypertensive agents have been implicated in control of VOC, nifedipine for example.

What is meant by “Hedgehog pathway” refers to Hedgehog (Hh) signal transduction. This pathway is initiated by the induction of the Hh precursor protein (45 kDa) in Hh-secreting cells, after which the precursor undergoes autocatalytic processing and modification. The precursor is cleaved to a 20 kDa N-terminal signal domain and a 25 kDa C-terminal catalytic domain. Subsequently, a cholesterol molecule is bound covalently to the carboxy terminus of the N-terminal domain, which is then secreted from the cytosol as a Hh ligand. On the surface of Hh-receiving cells there are two proteins of the pathway. One is Patched (Ptch), a twelve-pass transmembrane protein, interacts with the Hh ligand and the other is Smoothened (Smo), a seven pass transmembrane protein that is a signal transducer. In the absence of Hh ligands, Ptch interacts with Smo to inhibit its function and prevent activation of the downstream signaling cascade. Once the Hh ligand binds to Ptch along with Hh-interacting protein, Smo inhibition is released; this results in the activation of a downstream signaling cascade. This activation results in the release of a transcriptional factor GLI from a macromolecular complex on microtubules that includes the suppressor of fused, fused protein kinas A, GLI and possibly other components. GLI enters the nucleus and alters transcription of several genes, including those of the Hedgehog pathway. In vertebrates, Hh signaling activation requires cilium, a microtube based cell organelle.

“Antagonists of the Hedgehog pathway” refers to one or more molecules known to inhibit the Hedgehog family, including zerumbone epoxide, staurosporinone, 6-hydroxystaurosporinone, arcyriaflavin C, 5,6-dihyroxyarcyriaflavin A, physalin F, physalin B, cyclopamine, HPI-1, HPI-2, HPI-3, and HPI-4, arsenic trioxide (ATO), sodium arsenite, phenylarsine, GANT-58, GANT-61, and zerumbone (Kim et al., 2010, PNAS, 107:13432-37; Beauchamp et al. 2011, JCI 121:148-60; Lauth et al 2007, PNAS 104:8455-60; Hosoya et al., 2008, ChemBioChem 9:1082-92; Hyman et al, 2009, 106:14132-37; and Mas et al, 2010, Biochem. Pharm. 80:712-23, all hereby incorporated by reference in their entirety). Antagonists of the Hedgehog pathway also refers to drugs that are in clinical trials, including vismodegib (GDC-0449, Genentech), bevacizumab, gemcitabine, nab-paclitaxel, FOLFIR, FOLFOX, RO4929097, cixutumumab, cisplatin, etoposide, LDAC, decitabine, daunorubicin, cytarabin, rosiglitazone, goserelin, leuprolide, capecitabine, fluorouracil, leucovorin, oxaliplatin, irinotecan, diclofenac, BMS-833923 (XL139), IPI-926—Infinity Pharmaceuticals, Inc., LDE225, LEQ506—Novartis Pharmaceuticals, TAK-441 Millennium Pharmaceuticals, Inc., and PF-04449913—Pfizer, alone or in combination therapy. Inhibitors of cilium formation can also be used as Hh inhibitors.

As used herein, “arsenic compound” refers to a pharmaceutically acceptable form of arsenic trioxide (As203) or melarsoprol. Melarsoprol is an organic arsenic compound which can be synthesized by complexing melarsen oxide with dimercaprol or commercially purchased (Arsobal® by Rhone Poulenc Rorer, Collegeville, Pa.). Since the non-pharmaceutically formulated raw materials of the invention are well known, they can be prepared from well-known chemical techniques in the art. (See for example, Kirk-Othmer, Encyclopedia of Chemical Technology 4th ed. volume 3 pps. 633-655 John Wiley & Sons).

### Process for the Manufacture of Sterile Arsenic Trioxide Solution

The arsenic compounds of the invention may be formulated into sterile pharmaceutical preparations for administration to humans for treatment of HO or VOC. Compositions comprising a compound of the invention formulated in a compatible pharmaceutical carrier may be prepared, packaged, labeled for treatment of and used for the treatment of the indicated leukemia, lymphoma, or solid tumor.

In one aspect, the invention provides a method for the manufacture of a pharmaceutical composition comprising a therapeutic effective and non-lethal amount of arsenic trioxide (As203). Arsenic trioxide (raw material) is a solid inorganic compound that is commercially available in a very pure form. However, it is difficult to dissolve As203 in aqueous solution. Arsenic is present in solution in the +5 valence state (pentavalent) or the +3 valence state (trivalent). For example, potassium arsenite (KAs02; which is present in Fowler's solution) and salts of arsenious acid contain pentavalent arsenic. It is known that one form of arsenic is more toxic than the other. (Goodman & Oilman's The Pharmacological Basis of Therapeutics, 9th edition, chapter 66, 1660, 1997). A fresh solution of arsenic trioxide containing arsenic in the trivalent state will be gradually oxidized to pentavalent state if exposed to air for a prolonged period, and as a result of the accumulation of pentavalent arsenic, the relative toxicity of a solution of As203 will change over time. (Id.) Furthermore, it is observed that the total amount of arsenic in solution decreases over time. This loss of material is caused by the progressive conversion of arsenic in the solution into arsine (AsH3) which is a gaseous compound at room temperature. This is particularly problematic in pharmaceutical applications if the concentration of an active ingredient in the injected material cannot be controlled. It is also undesirable to allow arsine to escape from the solution into the atmosphere because arsine is also toxic.

An arsenic solution may be obtained using methods know in the art, for example by solubilizing solid high purity As203 in an aqueous solution at high pH using mechanical stirring and/or gentle heating, or by dissolving the solid compound overnight. Typically, a solution of 1 M As203 is obtained. To adjust the pH of the As203 solution, the solution may be diluted in water and the resulting solution neutralized with an acid such as hydrochloric acid, with constant stirring until the pH is about 8.0 to 8.5. The partially neutralized As203 solution may then be sterilized for example, by filtration (e.g., through a 0.22 μm filter), and stored in sterile vials.

To make a pharmaceutical composition that can be directly injected into a subject, the composition must be sterile, standard techniques known to the skilled artisan for sterilization can be used. See, e.g., Remington's Pharmaceutical Science. The pH of the partially neutralized As203 solution may be further adjusted to near physiological pH by dilution (10-100 fold) with a pharmaceutical carrier, such as a 5% dextrose solution. For example, 10 mL of a partially neutralized As203 solution can be added to 500 mL of a 5% dextrose solution to yield a final pH of about 6.5 to 7.5. The method of the invention reduces the oxidation of arsenic in solution. Pharmaceutical compositions containing arsenic trioxide manufactured by the method of the invention show improved stability and long shelf life.

### Formulation

The active ingredients of the invention are formulated into pharmaceutical preparations (e.g., together in a composition or separately to be used in a combination therapy) for administration to mammals for treatment of HO and/or VOC.

The following table provides a list of Hedgehog pathway antagonists, with dosage appropriate for current use of the drug in clinical trials for treating cancer. The skilled worker could adjust the dosage regime and mode of administration according to the status of the patient being treated for HO.

| Drug | Dosage |
|---|---|
| Vismodegib: | Visodegib: 150 mg orally (with or without food |
| Hedgehog | at the same time daily) |
| pathway | |
| antagonist; | |
| combination | |
| treatment | |
| includes | |
| Temozolomide, | Temozolomide: Dose in Cycle 1 is 150 mg/m2 |
| an alkylating | orally once daily for 5 days followed by 23 days |
| agent | without treatment. At the start of Cycle 2, the |
| | dose is escalated to 200 mg/m2 orally once daily |
| | for 5 days |
| GDC-0449 | Gelatine capsules of 150 mg, taken O.D. for 14 |
| | days |
| Bevacizumab | Bevacizumab - Intravenous repeating dose |
| | (dosage unknown) |
| Chemotherapy | Chemotherapy - Intravenous repeating dose |
| | (dosage unknown) |
| GDC-0449 | GDC-0449 - oral repeating dose (dose |
| | unknown) |
| | Placebo - oral repeating dose |
| Gemcitabine | 1. One cycle of Gemcitabine 1000 mg/m2 and |
| nab-Paclitaxel | nab-Paclitaxel 125 mg/m2 on days 1, 8, and 15 |
| GDC-0449 | (28 days cycle) then |
| | 2. Gemcitabine 1000 mg/m2 and nab-Paclitaxel |
| | 125 mg/m2 on days 1, 8, and 15 every 28 days |
| | cycle in combination with oral GDC-0449 150 |
| | mg daily |
| GDC-0449 | capsule, 150 mg, one pill daily, 18 months |
| GDC-0449 | GDC-0449 |
| | 150-mg capsule, once daily |
| Gemcitabine | Gemcitabine |
| | Administered intravenously at a dose of 1000 |
| | mg/m2 over 30 minutes on days 1, 8, 15 of a 4 |
| | week cycle (28 days) starting with cycle 2 |
| | (study day 29). |
| PF-04449913 | Escalating dose of PF-04449913 administered |
| | as tablets PO QD in 28-day cycles |
| GDC-0449 | 150 mg per day taken once daily for 28 days (1 |
| (Hedgehog | course). In the absence of unacceptable toxicity |
| antagonist) | or disease progression, treatment may continue |
| | for 26 courses (approximately 2 years). |
| GDC-0449 | Daily Oral repeating dose |
| | Three times weekly oral repeating dose |
| | Once weekly oral repeating dose |
| Bevacizumab | Bevacizumab |
| | Intravenous repeating dose |
| FOLFIR | FOLFIRI |
| | Intravenous repeating dose |
| FOLFOX | FOLFOX |
| | Intravenous repeating dose |
| GDC-0449 | GDC-0449 |
| | Oral repeating dose |
| Vismodegib | Vismodegib 150 mg was provided in hard |
| 150 mg | gelatin capsules. |
| GDC-0449 | 150 mg administered orally daily starting Day 8 |
| RO4929097 | RO4929097 administered single dose orally - |
| | Cycle 1, Day 1 and Days 1-3, 8-10 every 21 |
| | days starting Day 22 (Cycle 2, Day 1) Schedule |
| | B: RO4929097 administered single dose orally - |
| | Cycle 1, Day -2, -1, and 1 and PO Days 1-3, 8- |
| | 10 every 21 days starting Day 22 (Cycle 2, Day 1) |
| Cixutumumab | Cixutumumab - Given IV |
| GDC-0449 | GDC-0449 - Given IV |
| Cisplatin | Cisplatin - Given IV |
| etoposide | etoposide phosphate - Given IV |
| phosphate | |
| LDE-225 | Drug: LDE-225 |
| Gemcitabine | Phase 1 Stage: |
| | Four cycles of Gemcitabine 1000 mg/m2 on |
| | days 1, 8 and 15 in combination with escalating |
| | doses of LDE-225. |
| | Phase 2 Stage: |
| | 1. Arm A: Four cycles of gemcitabine 1000 |
| | mg/m2 on days 1, 8 and 15 in combination with |
| | LDE-225 at the recommended phase 2 dose. |
| | 2. Arm B: Four cycles of gemcitabine 1000 |
| | mg/m2 on days 1, 8 and 15. |
| | Drug: Gemcitabine |
| | Phase 1 Stage: |
| | Four cycles of Gemcitabine 1000 mg/m2 on |
| | days 1, 8 and 15 in combination with escalating |
| | doses of LDE-225. |
| | Phase 2 Stage: |
| | 1. Arm A: Four cycles of gemcitabine 1000 |
| | mg/m2 on days 1, 8 and 15 in combination with |
| | LDE-225 at the recommended phase 2 dose. |
| | 2. Arm B: Four cycles of gemcitabine 1000 |
| | mg/m2 on days 1, 8 and 15. |
| LDE225 | Dose escalation; given orally on a daily dosing |
| | schedule |
| PF-04449913 | PF-04449913: administered orally and |
| | continuously for 28-days. |
| LDAC | LDAC: administered at 20 mg SQ, BID on |
| | Days 1 through 10. |
| Decitabine | Decitabine: given at 20 mg/m2 over an hour |
| | infusion for 5-days |
| Daunorubicin | Daunorubicin: given using 60 mg/m2 for 3-days |
| Cytarabine | Cytarabine: 100 mg/m2 on days 1 through 7 |
| PF-04449913 | Escalating doses of PF-04449913 administered |
| | as tablets PO QD continuously in 28 day cycles |
| FOLFOX | FOLFOX regimen: Given IV |
| regimen | |
| GDC-0449 | GDC-0449: Given orally |
| Fluorouracil | Fluorouracil: Given IV |
| leucovorin | leucovorin calcium: Given IV |
| calcium | |
| oxaliplatin | oxaliplatin: Given IV |
| IPI-926 | IPI-926 and Cetuximab |
| Cetuximab | Patients will receive Cetuximab IV every week. |
| | Starting on Day 15 of the first cycle, Patients |
| | will take IPI-926by mouth every day. |
| Vismodegib | 150 mg orally once daily until disease |
| (GDC-0449) | progression; intolerable toxicity, most probably |
| | attributable to vismodegib; or withdrawal from |
| | the study. |
| GDC-0449 | oral dosage administration, intravenous dosage |
| | administration |
| Vismodegib | oral repeating dose |
| GDC-0449 | Given orally once daily for 7 days prior to |
| | surgery |
| IPI-926 | 160 mg, 130 mg or 110 mg oral daily dosing, |
| | until progressive disease or intolerability to |
| | study treatments or withdrawal of ICF |
| LDE225 | Etoposide will be dosed at 120 mg/m2 daily on |
| Etoposide | days 1, 2, and 3; cisplatin will be dosed at |
| Cisplatin | 60 mg/m2 on day 1; LDE225 will begin on cycle |
| | I/day 1 and will be dosed daily. As explained |
| | above, two dose levels of LDE225 will be |
| | evaluated, with plans for one dose de-escalation |
| | if necessary. Each cycle will consist of 3 weeks |
| | (21 days), and be repeated 6 times, unless the |
| | patient develops progressive disease or |
| | unacceptable toxicity. “After 6 cycles of |
| | LDE225 with etoposide and cisplatin, patients |
| | will be able to continue taking LDE225 as |
| | maintenance until progression of disease, |
| | unacceptable toxicity or withdrawal of consent.” |
| GDC-0449 | Gemcitabine hydrochloride IV over 30 minutes |
| gemcitabine | on days 1, 8, and 15 and oral hedgehog |
| hydrochloride | antagonist GDC-0449 once daily on days 1-28. |
| | Courses repeat every 28 days in the absence of |
| | disease progression or unacceptable toxicity |
| GDC-0449 | No dosage information provided |
| oral | |
| contraceptive | |
| rosiglitazone | |
| GDC-0449 | oral systemic Hedgehog antagonist GDC-0449 |
| | once on day 1 and then once or twice daily |
| | beginning on day 8 and continuing for up to 49 |
| | weeks in the absence of disease progression or |
| | unacceptable toxicity. |
| BMS-833923 | Capsules, Oral, 30 mg starting; dose escalation, |
| (XL139) | Once daily, 37 days; additional days if receiving |
| | benefit |
| GDC-0449 | 150 mg, 300 mg, or 450 mg once daily for 28 |
| | days (1 course). Treatment may continue for 13 |
| | courses (approximately 1 year) in the absence of |
| | unacceptable toxicity or disease progression. |
| IPI-926 plus | Daily IPI-926 (oral) at 160 mg plus gemcitabine |
| gemcitabine | (infusion) at 1000 mg/m2 once weekly for 3 |
| | weeks of a 28 day cycle |
| GDC-0449 | 150 mg GDC-0449 on Day 1 either after fasting |
| | or high or low fat meal (as assigned) followed |
| | by week of no drug |
| | 150 mg GDC-0449 once daily in 28 day cycle |
| | taken either after fasting or low fat meal |
| GDC-0449 | (PO) once daily on days 1-28. Treatment repeats |
| | every 28 days for up to 11 courses in the |
| | absence of disease progression or unacceptable |
| | toxicity. |
| goserelin | goserelin acetate |
| acetate | Given intramuscularly or subcutaneously |
| leuprolide | leuprolide acetate |
| acetate | Given intramuscularly or subcutaneously |
| vismodegib | vismodegib |
| | Given orally |
| TAK-441 | oral tablet, single-dose administration on Day 1, |
| | followed by a 1-week washout period during |
| | which pharmacokinetics is assessed |
| | Continuous daily dosing on Days 8 through 28 |
| | in Cycle 1 |
| | In subsequent cycles, continuous daily dosing |
| | over 21 days, repeated continuously |
| BMS-833923 | BMS-833923 |
| | Capsule, Oral, Starting dose 30 mg, Once daily, |
| | continuous until discontinuation from study |
| Cisplatin | Cisplatin |
| | Vial, intravenous (IV), 80 mg/m² IV, Once |
| | every 21 days, 1 day per cycle until |
| | discontinuation from study |
| Capecitabine | Capecitabine |
| | Tablets, Oral, 1000 mg/m², twice a day (BID), |
| | 14 days per cycle, until discontinuation from |
| | study |
| NA | NA |
| NA | NA |
| IPI-926 | Oral daily dosing |
| LDE225 | Oral daily dosing with dose-escalation |
| LDE225 | Oral daily dosing |
| vismodegib | Oral daily dosing on days 1-28. Treatment |
| | repeats every 28 days for up to 26 courses in the |
| | absence of disease progression or unacceptable |
| | toxicity. |
| LEQ506 | Oral daily dosing with dose-escalation |
| LDE225 | Oral daily dosing for 12 weeks |
| LDE225 and | No dosage information available |
| BKM120 | |
| IPI-926 | Oral dosing |
| LDE225 | LDE225 200-800 mg daily orally. |
| Fluorouracil | Fluorouracil 2400 mg IV every other week. |
| Leucovorin | Irinotecan 180 mg/m2 IV every other week. |
| Oxaliplatin | Oxaliplatin 85 mg/m2 IV every other week. |
| Irinotecan | Leucovorin 400 mg/m2 IV every other week. |
| BMS-833923 | BMS-833923: Capsule, Oral, starting dose 30 |
| | mg, once daily, continuous |
| Carboplatin | Carboplatin: Vial, Intravenous (IV), dose to |
| | yield 5 mg/mL - min, once every 21 days, 1 day |
| | per cycle up to 4 cycles |
| Etoposide | Etoposide: Vial, Intravenous (IV), 100 |
| | mg/m²/dose, days 1, 2, & 3 of each 21 day |
| | cycle, 3 days per cycle for up to 4 cycles |
| NA | NA |
| Diclofenac | Diclofenac: Application on the lesion 2 times a |
| | day 8 weeks. |
| Diclofenac + | Diclofenac + Calcitriol: Application on the |
| Calcitriol | lesion 2 times a day, both ointments, 8 weeks. |
| Calcitriol | Calcitriol: Application on the lesion, 2 times a |
| | day, 8 weeks. |
| RO4929097 | RO4929097: Given orally |
| vismodegib | Vismodegib: Given orally |

For oral administration of arsenic compounds, the pharmaceutical preparation can be in liquid form, for example, solutions, syrups or suspensions, or can be presented as a drug product for reconstitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, or fractionated vegetable oils); and preservatives (e.g., methyl or propyl-p-hydroxybenzoates or sorbic acid). The pharmaceutical compositions can take the form of, for example, tablets or capsules prepared by conventional means with pharmaceutically acceptable excipients such as binding agents (e.g., pregelatinized maize starch, polyvinyl pyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets can be coated by methods well-known in the art.

Preparations for oral administration can be suitably formulated to give controlled release of the active compound. For oral administration, the compositions can take the form of tablets or lozenges formulated in conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

The therapeutic agents consisting of arsenic compounds can be formulated for parenteral administration by injection, e.g., by bolus injection or continuous infusion. Such formulations are sterile. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents. Alternatively, the active ingredient can be in powder form for constitution with a suitable vehicle, e.g., sterile pyrogen-free water, before use.

The compounds can also be formulated in rectal compositions such as suppositories or retention enemas, e.g., containing conventional suppository bases such as cocoa butter or other glycerides.

In addition to the formulations described previously, the compounds can also be formulated as a depot preparation. Such long acting formulations can be administered by implantation (for example, subcutaneously or intramuscularly) or by intramuscular injection. Thus, for example, the compounds can be formulated with suitable polymeric or hydrophobic materials (for example, as emulsion in acceptable oils) or ion exchange resins, or as sparingly soluble derivatives, for example, as a sparingly soluble salt. Liposomes and emulsions are well known examples of delivery vehicles or carriers for hydrophilic drugs.

The pharmaceutical preparations can, if desired, be presented in a pack or dispenser device which can contain one or more unit dosage forms containing the active ingredient. The pack can for example comprise metal or plastic foil, such as a blister pack. The pack or dispenser device can be accompanied by instructions for administration.

Any suitable mode of administration may be used in accordance with the present invention including but not limited to parenteral administration such as intravenous, subcutaneous, intramuscular and intrathecal administration; oral, and intranasal administration, and inhalation. The mode of administration will vary according to the degree of HO or VOC, and the condition of the human.

The pharmaceutical compositions to be used may be in the form of sterile aqueous or organic solutions; colloidal suspensions, caplets, tablets and cachets.

In accordance with the present invention, arsenic trioxide or melarsoprol compounds can be used alone or in combination with other known therapeutic agents or techniques to either improve the quality of life of the patient, or to treat HO or VOC. For example, the arsenic compounds can be used before, during or after the administration of one or more known anti-inflammatory agents

### Method of Administration

It will be recognized by one of skill in the art that the content of the active ingredients in the pharmaceutical preparations of this invention can vary quite widely depending upon numerous factors such as, the desired dosage and the pharmaceutically acceptable carrier being employed. The table above lists Hedgehog pathway antagonists and modes of administration for each.

For administration of an arsenic compound, the dosage amount will usually be in the range of from about 0.1 mg/kg to about 100 mg/kg, in certain embodiments from about 1.0 to about 50 mg/kg, in other embodiments from about 2.5 to about 25 mg/kg, and in other embodiments from about 3 to about 15 mg/kg.

The invention also provides kits for carrying out the therapeutic regimens of the invention. Such kits comprise in one or more containers therapeutically effective amounts of the arsenic compounds in pharmaceutically acceptable form. The arsenic compound in a vial of a kit of the invention may be in the form of a pharmaceutically acceptable solution, e.g., in combination with sterile saline, dextrose solution, or buffered solution, or other pharmaceutically acceptable sterile fluid. Alternatively, the complex may be lyophilized or desiccated; in this instance, the kit optionally further comprises in a container a pharmaceutically acceptable solution (e.g., saline, dextrose solution, etc.), preferably sterile, to reconstitute the complex to form a solution for injection purposes.

In another embodiment, a kit of the invention further comprises a needle or syringe, preferably packaged in sterile form, for injecting the complex, and/or a packaged alcohol pad. Instructions are optionally included for administration of arsenic compounds by a clinician or by the patient.

Desirable blood levels may be maintained by a continuous infusion of an arsenic compound as ascertained by plasma levels. It should be noted that the attending physician would know how to and when to terminate, interrupt or adjust therapy to lower dosage due to toxicity, or bone marrow, liver or kidney dysfunctions. Conversely, the attending physician would also know how to and when to adjust treatment to higher levels if the clinical response is not adequate (precluding toxic side effects).

Again, any suitable route of administration may be employed for providing the patient with an effective dosage of an arsenic compound. For example, oral, transdermal, iontophoretic, parenteral (subcutaneous, intramuscular, intrathecal and the like) may be employed. Dosage forms include tablets, troches, cachet, dispersions, suspensions, solutions, capsules, patches, and the like. (See, Remington's Pharmaceutical Sciences.)

The pharmaceutical compositions of the present invention comprise an arsenic compound as the active ingredient, pharmaceutically acceptable salt thereof, and may also contain a pharmaceutically acceptable carrier, and optionally, other therapeutic ingredients, for example all trans retinoic acid. The term “pharmaceutically acceptable salts” refers to salts prepared from pharmaceutically acceptable non-toxic acids and bases, including inorganic and organic acids and bases.

The pharmaceutical compositions include compositions suitable for oral, mucosal routes, transdermal, iontophoretic, parenteral (including subcutaneous, intramuscular, intrathecal and intravenous), although the most suitable route in any given case will depend on the nature and severity of the condition being treated.

In the case where an intravenous injection or infusion composition is employed, a suitable dosage range for use is, e.g., from about one to about 40 mg arsenic trioxide total daily; about 0.001 to about 10 mg arsenic trioxide per kg body weight total daily, or about 0.1 to about 10 mg melarsoprol per kg body weight total daily.

In addition, the arsenic carrier could be delivered via charged and uncharged matrices used as drug delivery devices such as cellulose acetate membranes, also through targeted delivery systems such as fusogenic liposomes attached to antibodies or specific antigens.

In practical use, an arsenic compound can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including tablets, capsules, powders, intravenous injections or infusions). In preparing the compositions for oral dosage form any of the usual pharmaceutical media may be employed, e.g., water, glycols, oils, alcohols, flavoring agents, preservatives, coloring agents, and the like; in the case of oral liquid preparations, e.g., suspensions, solutions, elixirs, liposomes and aerosols; starches, sugars, micro-crystalline cellulose, diluents, granulating agents, lubricants, binders, disintegrating agents, and the like in the case of oral solid preparations e.g., powders, capsules, and tablets. In preparing the compositions for parenteral dosage form, such as intravenous injection or infusion, similar pharmaceutical media may be employed, e.g., water, glycols, oils, buffers, sugar, preservatives and the like know to those skilled in the art. Examples of such parenteral compositions include, but are not limited to Dextrose 5% w/v, normal saline or other solutions. The total dose of the arsenic compound may be administered in a vial of intravenous fluid, e.g., ranging from about 2 ml to about 2000 ml. The volume of dilution fluid will vary according to the total dose administered. For example, arsenic trioxide supplied as a 10 ml aqueous solution at 1 mg/ml concentration is diluted in 10 to 500 ml of 5% dextrose solution, and used for intravenous infusion over a period of time ranging from about ten minutes to about four hours.

### EXAMPLES

### Example 1

### Removal of Gαₛ from Mesenchymal Cells Leads to HO

All mice used in these examples have been previously described in the literature, including: Gαₛ^{flox} (Chen, M., et al., 2005, J Clin Invest 115, 3217-27), Prx1-cre (Logan, M., et al., 2002, Genesis 33, 77-80), Dermo1-cre Yu, K., et al., 2003, Development 130, 3063-74), Ap2-cre (Nelson, et al., 2004, Dev Biol 267, 72-92), and Ptch1^{flox} (Mak, K.K., et al., 2006, Development 133, 3695-3707).

Alizarin Red-Alcian Blue Staining:

Embryos were skinned and placed in 100% EtOH overnight to fix. Embryos were then placed in staining solution for 2 days (50 mL staining solution=2.5 mL 0.3% Alcian Blue, 2.5 mL 0.1% Alizarin Red, 2.5 mL 100% glacial acetic acid, 42.5 mL 70% EtOH). Embryos were rinsed with water, then placed in 1% KOH until destained, and finally in 80% glycerol for storage.

Von Kossa Staining:

Tissue sections were deparaffinized and hydrated in distilled water. silver hydrate, 5%, was added to slides and place under a 60-watt lamp for 1 hour. Slides were rinsed three times in distilled water before adding 5% sodium thiosulfate for 5 minutes. Slides were rinsed three times in distilled water, and counterstained with nuclear fast red for 5 minutes. Slides were again rinsed three times in distilled water, dehydrated, cleared and put under a coverslip.

Osterix Immunohistochemistry:

Tissue sections were deparaffinized and hydrated in distilled water. Slides were placed into boiling 10 mM citrate pH6 for 15 minutes, and then placed at room temperature for 15 minutes. Slides were placed in 3% water/MeOH for 15 minutes. Slides were equilibrated in phosphate buffered saline with 0.1% Tween-20 (PBS-T) then block for 1 hour with 5% normal goal serum in PBS-T. Rabbit anti-osterix antibody (Abcam; ab22552) was added at 1:1000 and incubated overnight at 4° C. Slides were washed and detected using the anti-rabbit ABC elite kit (Vector labs; PK-6101) and DAB tablets (Sigma-Aldrich; D4293). Slides were counterstained with nuclear fast red and Alcian Blue, then dehydrated, cleared, and put under a coverslip.

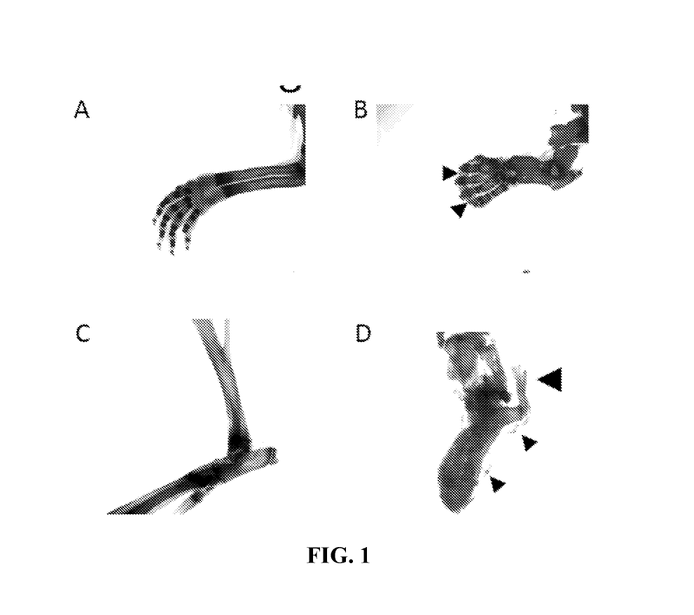

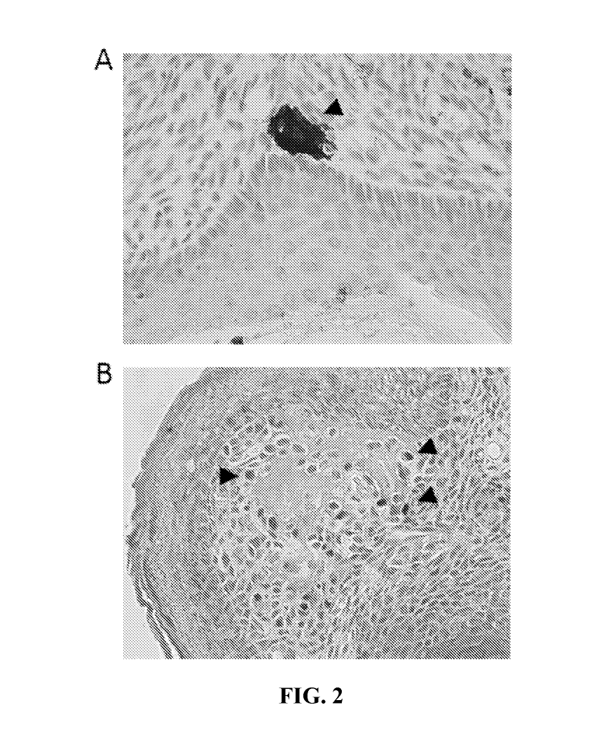

Using the Prx1-cre, Gαₛ was removed in undifferentiated limb bud mesenchyme. While the limb pattern was largely normal, Prx1-cre+; Gαₛ^{flox/-} mice were born with a soft tissue syndactyly (webbing between the digits) and a progressive form of HO. In the skeletal preparations examined, bone was stained red and cartilage was stained blue. HO first appeared by Alizarin Red staining at embryonic day 16.5 and was clearly visible by postnatal day 4 in Prx1-cre+; Gαₛ^{flox/-} mice (arrows, FIGS. 1A, 1B). HO progressed leading to bone and joint fusions and ossification of tendons (arrowhead, FIGS. 1C, 1D). Von Kossa staining further confirmed the presence of subcutaneous mineralized tissue in Prx1-cre+; Gαₛ^{flox/-} mice (arrow, FIG. 2A), which was never seen in control mice. Cells surrounding these ossicles stained positive for Osterix, a marker of early osteoblasts, demonstrating HO was generated by bone forming cells (arrows, FIG. 2B). A similar phenotype was also obtained when Gαₛ was removed using either the Dermo1-cre or Ap2-cre, which also excised in mesenchymal tissues. This is consistent with a requirement for Gαₛ to suppress HO formation in mesenchymal tissues.

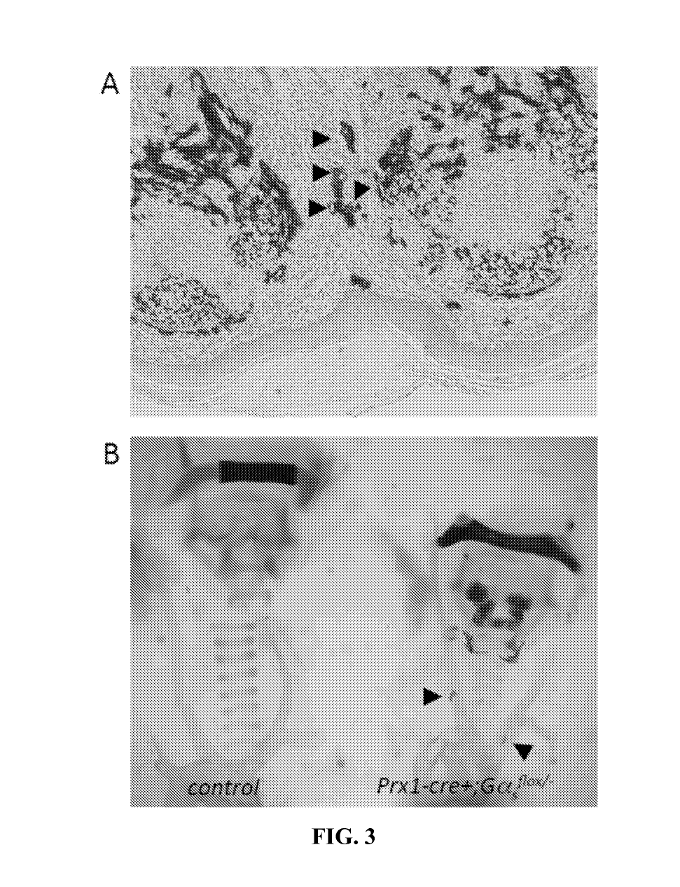

Loss of Gαₛ was also associated with the formation of vascular calcification/ossification. In Prx1-cre+; Gαₛ^{flox/-} mice ossification is frequently seen surrounding blood vessels between the digits (arrows, FIG. 3A). In Dermo1-cre+; Gαₛ^{flox/-} mice, mineralization is also seen around the great vessels (arrows, FIG. 3B) (Thomas, et al., 2002, Orthop Clin North Am 23, 347-58).

These results demonstrate that loss of Gαₛ promotes HO formation both in soft tissues and around blood vessels.

### Example 2

### Loss of Gαₛ Leads to Activation of the Hh Pathway

The formation of HO indicated inappropriate upregulation of a pro-osteogenic pathway (e.g. BMP, Wnt, and/or hedgehog) outside the skeleton. As Gαₛ is a physiologically important activator of protein kinase A (PKA) and PKA is a potent inhibitor of Hedghehog (Hh) signaling (Jiang, et al., 1995, Cell 80, 563-572), loss of Gαₛ is likely associated with an upregulation of Hh that was driving HO formation. Accordingly, Hh signaling would be elevated prior to HO formation and overlap in its tissue expression. Quantitative reverse transcriptase polymerase chain reaction (qRT-PCR) was performed on RNA isolated from control and Prx1-cre+; Gαₛ^{flox/-} mutant limb tissue at E14.5, prior to HO formation. The following primers were used for qRT-PCR:

Actin:
Forward
5′-CAC AGC TTC TTT GCA GCT CCT T-3′,
Reverse
5′-CGT CAT CCA TGG CGA ACT G-3′;
tubulin:
Forward
5′- CAA CGT CAA GAC GGC CGT GTG-3′,
Reverse
5′-GAC AGA GGC AAA CTG AGC ACC-3′;
Gαₛ:
Forward
5′- GCA GAA GGA CAA GCA GGT CT-3′,
Reverse
5′-CCC TCT CCG TTA AAC CCA TT-3′;
Ptch1:
Forward
5′-CTC TGG AGC AGA TTT CCA AGG-3′,
Reverse
5′-TGC CGC AGT TCT TTT GAA TG-3′;
Gli1:
Forward
5′-GAA AGT CCT ATT CAC GCC TTG A-3′,
Reverse
5′-CAA CCT TCT TGC TCA CAC ATG TAA G-3′;
HIP:
Forward
5′-GGG AAA AAC AGG TCA TCA GC-3′,
Reverse
5′-ATC CAC CAA CCA AAG GGC-3′.
Osx:
Forward
5′- CCC ACT GGC TCC TCG GTT CTC TCC -3′,
Reverse
5′-GCTBGAA AGG TCA GCG TAT GGC TTC -3′;
Colla1:
Forward
5′- CAC CCT CAA GAG CCT GAG TC -3′,
Reverse
5′- GTT CGG GCT GAT GTA CCA GT -3′;
Alkaline Phosphatase:
Forward
5′- CAC GCG ATG CAA CAC CAC TCA GG -3′,
Reverse
5′- GCA TGT CCC CGG GCT CAA AGA -3′;
BSP:
Forward
5′- TAC CGG CCA CGC TAC TTT CTT TAT -3′,
Reverse
5′- GAC CGC CAG CTC GTT TTC ATC C -3′;
Oc:
Forward
5′- ACC CTG GCT GCG CTC TGT CTC T -3′,
Reverse
5′- GAT GCG TTT GTA GGC GGT CTT CA -3′.

Relative expression was quantified using the 2^{-AAct} method (Livak, et al., 2001, Methods 25, 402-08).

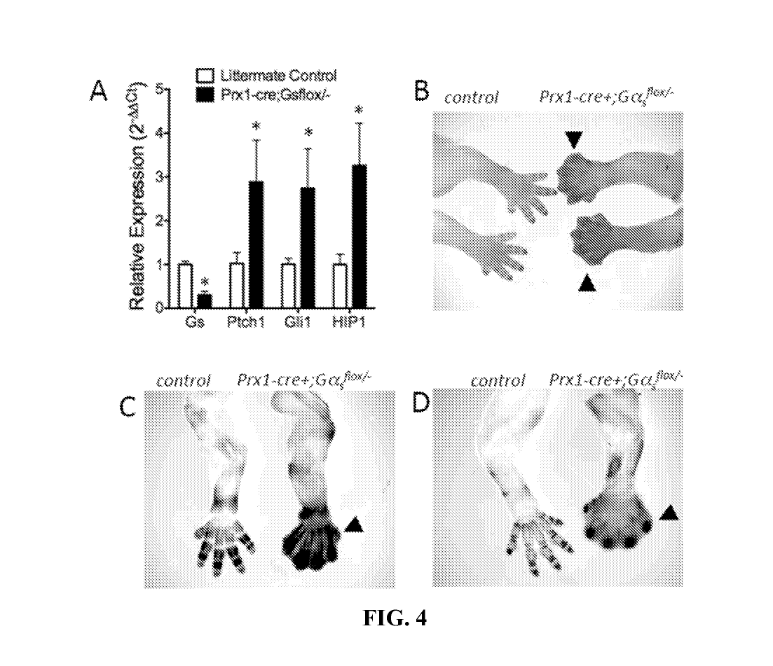

Prx1-cre+; Gαₛ^{flox/-} mice showed a reduction in Gαₛ mRNA (FIG. 4A). Importantly, mutant mice also showed an approximately 3-fold upregulation of mRNA for Patched1, Gli1 and hedgehog interacting protein (HIP) (FIG. 4A). These three most commonly used markers of Hh pathway activation provide strong evidence that Hh signaling was upregulated in these mutant mice that go on to develop HO. Since elevated Hh is driving HO formation, expression of these markers overlap with the interdigit regions where HO is more prevalent. In situ hybridization was performed on control and mutant forelimbs with antisense digoxygenin-labeled probes for Ptch1, Gli1 and HIP, using whole mount in situ hybridization using standard techniques. The results showed both increased staining in the mutant and overlap with the interdigit areas, where HO was first seen (FIGS. 4B, 4C, and 4D).

Elevation in the protein level of the Gli3 full-length form was also seen during Hh pathway activation (Wang, et al. 2000, Cell 100, 423-34). Western blot analysis was performed on limb tissue isolated from E18.5 control and mutant mice. The presence of the Gli3 full-length form was assayed. This product has a molecular weight of ˜190 kDa versus the repressor form which has an apparent molecular weight of ˜83 kDa. Western blotting was performed using standard techniques using a rabbit anti-Gli3 antibody (Dr. Susan Mackem (NIH/NCI)), a rabbit anti-Gαₛ (Dr. Lee Weinstein (NIH/NIDDK)) and a rat anti-α-tubulin (Sigma).

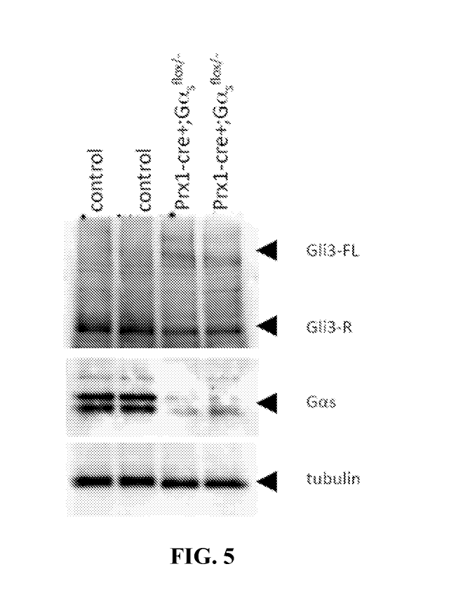

Western blotting performed with an antibody raised against Gli3 demonstrated that Gli3 full-length form (Gli3-FL) was present at higher levels and Gli3 repressor form (Gli3-R) at lower levels in the mutant relative to littermate controls (FIG. 5). Western blots were also performed against Gαₛ to confirm its reduction in the mutant and against tubulin to demonstrate equal loading.

All of these data are consistent with the Hh pathway being upregulated in the mutant mice that form HO, which suggests inhibiting the Hh pathway may be an important treatment for HO.

### Example 3

### Ptch1 Interacts Genetically with Gαₛ

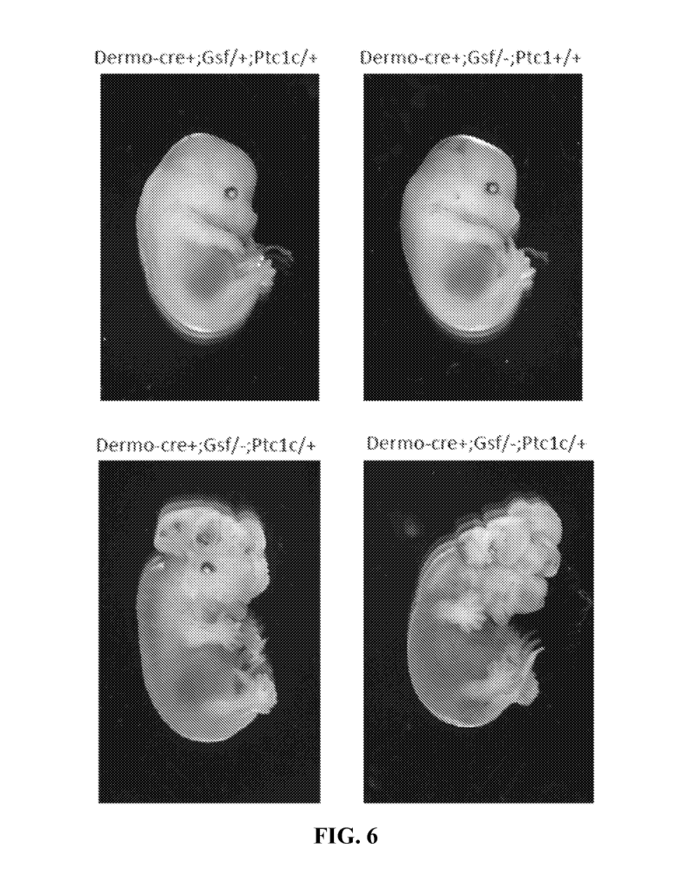

Ptch1 is an inhibitor of the Hh pathway and loss of both copies of the Ptch1 gene leads to elevated Hh signaling and embryonic lethality (Goodrich, et al., 1997, Science 277, 1109-13). Mice lacking one copy of Ptch1 are viable and highly sensitized to further increases in Hh signaling. To show Gαₛ is a biologically important regulator of the Hh pathway, the genetic interaction of Gαₛ and Ptch1 was measured. male Dermo1-cre+; Gαₛ^{flox/-};Ptch1^{flox/-} mice were mated to Gαₛ^{flox/flox} female mice. If Gαₛ and Ptch1 do not interact genetically then Dermo1-cre+; Gαₛ^{flox/-};Ptch1^{flox/+} and Dermo1-cre+; Gαₛ^{flox/-};Ptch1^{1+/+} would look similar. If Gαₛ and Ptch1 do interact genetically then Dermo1-cre+; Gαₛ^{flox/-};Ptch1^{flox/-} mice would look more severe than either Dermo1-cre+; Gαₛ^{flox/-};Ptch1^{1+/+} or Dermo1-cre+; Gαₛ^{flox/-};Ptch1^{flox/+} mice. At E18.5, no Dermo1-cre+; Gαₛ^{flox/-};Ptch1^{flox/+} mice were observed, likely because these mice died prior to this stage. Dermo1-cre+; Gαₛ^{flox/-};Ptch1^{flox/+} mice were present in the correct Mendellian ratio at E13.5, and presented with severe skeletal defects including polydactyly, exencephaly and craniofacial defects (FIG. 6).

These data further demonstrate that Gαₛ and Ptch1 interact which confirms that elevated Hh signaling is associated with multiple pathologic phenotypes, including HO.

### Example 4

### Removal of Gαₛ from Mesenchymal Cells Leads to Increased Osteoblast Differentiation

HO requires an inappropriate increase in bone forming cells (osteoblasts) The role of Gαₛ in promoting osteoblastic differentiation was tested. Bone marrow mesenchymal cells (BMMC) (also called mesenchymal stem cells) were isolated from Gαₛ^{flox/flox} mice. BMMC were isolated by flushing the bone marrow cavity of 6 week old mice and plating cells in Alpha-MEM, 20% FBS, 100 U/mL penicillin, 100 μg/mL streptomycin, 2 mM glutamine. Prior to reaching confluence cells were treated with a Cre- or GFP-containing adenovirus. Upon reaching confluence cells were switched to osteogenic media (DMEM, 10% lot-selected FBS, 100 U/mL penicillin, 100 ng/mL streptomycin, 2 mM glutamine, 10-4M L-ascorbic acid 2-phosphate and 10 mM α-glycerol phosphate). These cells were infected with a Cre-containing adenovirus to remove Gαₛ and cultured under conditions that favor osteogenic differentiation.

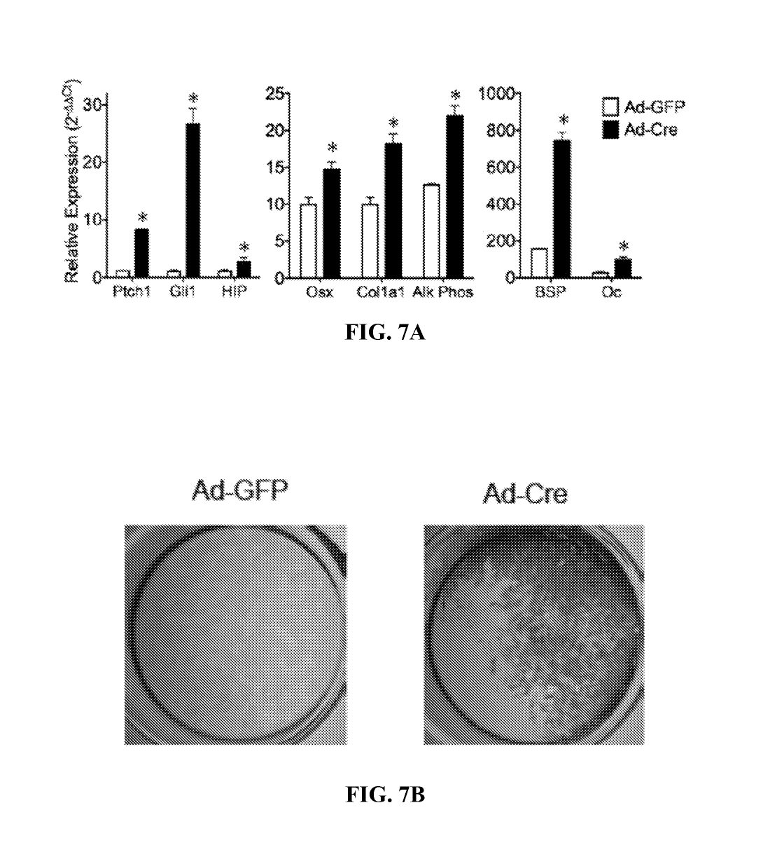

Upon reaching confluence in vitro, a significant rise in Hh target genes in these cells was found (FIG. 7). This was followed by an associated increase in expression of markers of osteoblast differentiation (osterix (Osx), collagen 1a1 (Col1a1), alkaline phosphatase (Alk Phos), bone sialoprotein (BSP), osteocalcin (OC)).

This result confirms the role of Gαₛ in promoting osteoblastic differentiation.

### Example 5

### Treatment with ATO In Vitro Reduces Hh Levels

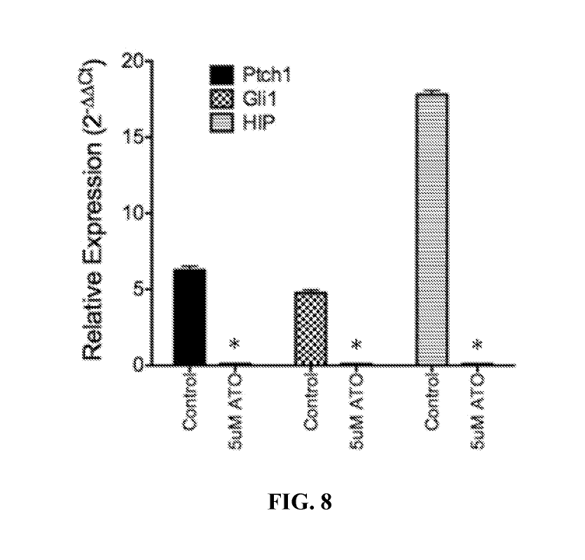

The role of inhibitors of Hh signaling in decreasing the elevated Hh signaling observed in Prx1-cre+; Gαₛ^{flox/-} mutant mice. Limb culture experiments were performed in which the limbs from living embryos at E14.5 were removed and cultured in vitro for 4 days. Limb culture was performed by using BGJB culture media supplemented with 0.2% bovine serum albumin (Sigma Aldrich) and forskolin (Sigma Aldrich), cyclopamine (BIOMOL), or ATO (Sigma Aldrich). ATO was prepared by placing 50 mg of ATO in the bottom of a 50 mL conical tube and dissolving with 1 mL of 1N NaOH. 48 mL of PBS was then added to the tube and 0.82 mL of 1.2N HCl to adjust pH to 7.2. Media was changed daily following addition of culture media. In culture, the limbs were cultured with vehicle or ATO and assayed for reduced Hh pathway activation by qRT-PCR. Mutant mice demonstrated elevated Hh signaling, by increased expression of Ptch1, Gli1 and HIP mRNA (FIG. 8).

Treatment of Prx1-cre+; Gαₛ^{flox/-} mutant limbs with the Hh pathway antagonist ATO led to a dramatic reduction in the Hh pathway activation. This result is consistent with a role of ATO in blocking HO formation.

### Example 6

### HO Development with Ad-Cre-Driven Loss of Gαₛ

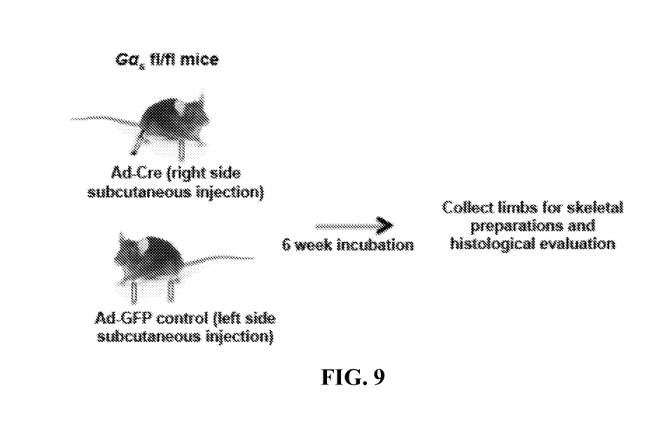

HO development in adult mice was studied by using adenovirus cre recombinase (Ad-Cre)-driven loss of Gαₛ (FIG. 9): Ad-Cre injections (100 μL of 1:10 diluted virus in PBS) were administered subcutaneously above Gα_{sflox/flox} mouse right limbs, while the control Adenovirus GFP (Ad-GFP) subcutaneous injections were given subcutaneously over Gα_{sflox/flox} mouse left limbs when these mice were 1 months old. 6 weeks following the adenovirus injection, the mice were sacrificed and limbs (with intact subcutaneous tissue) were collected for skeletal preparations and histological evaluations.

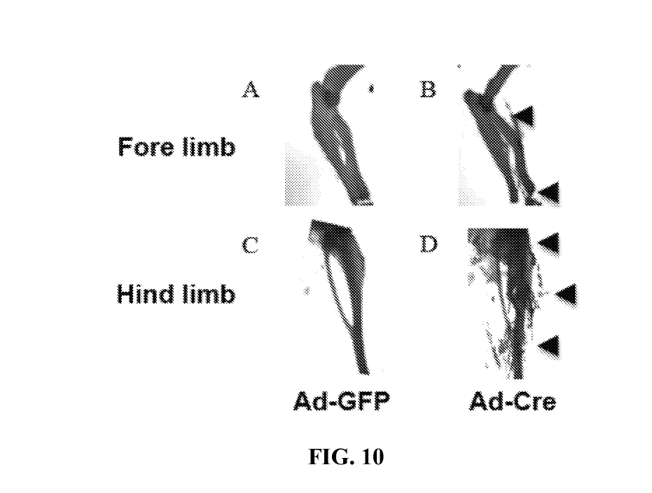

Results showed ectopic bone forms over the right forelimb and hind limb following Ad-Cre-mediated loss of Gαₛ in Gα_{sflox/flox} mice and not on the control side left limbs with Ad-GFP injection (FIG. 10). Alizarin Red stains bone and Alcian Blue stains cartilage. FIG. 10A shows control forelimb from a 10 week old Gα_{sflox/flox} mouse, 6 weeks after Ad-GFP administration, shows normal limb architecture. FIG. 10B shows a mutant limb from a 10 weeks old Gα_{sflox/flox} mouse, six weeks after Ad-Cre administration with profound HO (arrowhead) forming in soft tissues. FIG. 10C shows a control hind limb from a 10 weeks old Gα_{sflox/flox} mouse, 6 weeks after Ad-GFP administration, shows normal limb architecture. FIG. 10D shows a mutant limb from a 10 weeks old Gα_{sflox/flox} mouse, 6 weeks after Ad-Cre administration with profound HO (arrowhead) forming in soft tissues over the endogenous bone.

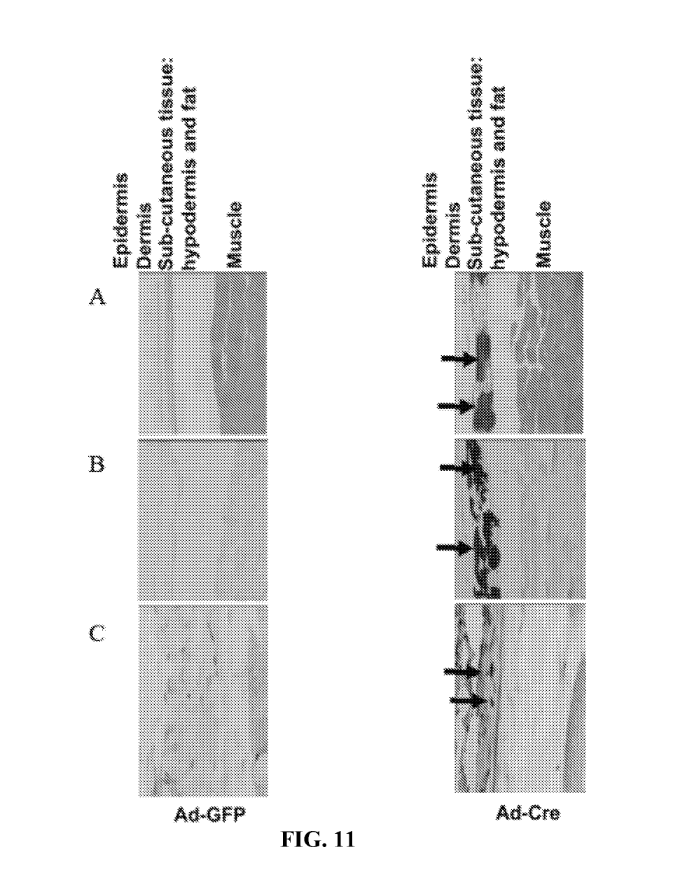

HO in adult loss of Gαₛ in Gα_{sflox/flox} mice is shown in FIG. 11. (A) shows H and E staining suggests the presence of mineralization in subcutaneous tissue about right limbs Gα_{sflox/flox} mice (arrow), which received Ad-Cre injection, while no subcutaneous mineralization observed on the control left side in Gα_{sflox/flox} mouse left limb, following Ad-GFP injection. A nuclear fast red counterstain allows the visualization of tissue architecture. (B) shows Von Kossa staining demonstrates the presence of mineralized tissue in subcutaneous tissue above right limbs Gα_{sflox/flox} mice (arrow), which received Ad-Cre injection, while no subcutaneous mineralization observed on the control left side in Gα_{sflox/flox} mouse left limb, following Ad-GFP injection. A nuclear fast red counterstain allows the visualization of tissue architecture. (C) shows immunohistochemistry for the early osteoblastic marker osterix demonstrates its presence (brown nuclear staining; arrows) in cells in the subcutaneous ossicle. A nuclear fast red counterstain allows the visualization of tissue architecture.

### Example 7

### Effects of Removal of Gαₛ from BMMC

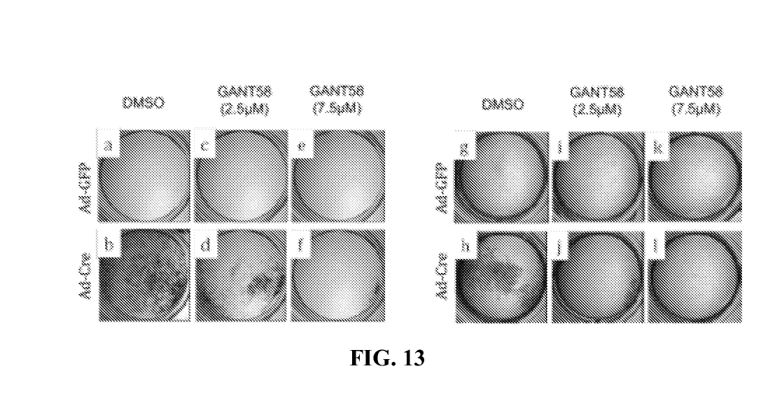

Removal of Gαₛ from bone marrow mesenchymal cells (BMMC) promotes osteoblastic differentiation, which is inhibited by Hh antagonist, GANT-58 treatment in a dose-dependent manner (FIG. 13). BMMCs were isolated from Gα_{sflox/flox} mice and infected with a Cre-containing adenovirus (ad-Cre) to remove Gαₛ or GFP-containing adenovirus (Ad-GFP) control. Cells were grown to confluence and placed in osteogenic media for 7 days. Von Kossa staining analysis demonstrates an increase in tissue mineralization at confluence following removal of Gαₛ (panel b) as compared to Ad-GFP treatment (panel a), which is inhibited by GANT-58 in a dose-dependent manner (panels d and f compared to panel b). No effect of GANT-58 observed in Ad-GFP treated cells (panels c and e compared to a). Similar inhibition of osteoblast differentiation was observed by Alizarin red staining (panels j and l compared to h).

### Example 8

### Smoothed is Associated with HO

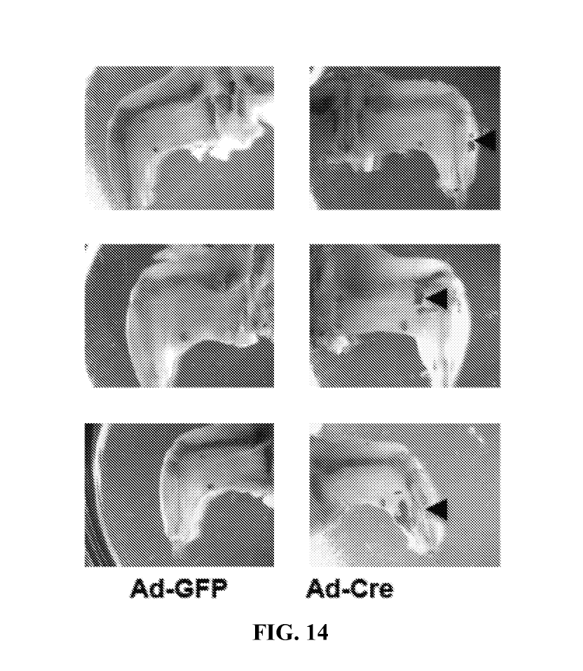

HO in with adult gain of function of hedgehog effector protein Smoothed, in Gt(ROSA)26Sortm1(Smo/YFP)Amc/J mice following Ad-Cre subcutaneous injection. Ectopic bone forms over the right forelimb and hind limb following Ad-Cre-mediated loss of Gαₛ in Gα_{sflox/flox} mice and not on the control side left limb with Ad-GFP injection. Alizarin Red stains bone and Alcian Blue stains cartilage (FIG. 14). (A) A control forelimb from a 10 weeks old Gt(ROSA)26Sortm1(Smo/YFP)Amc/J mouse, 8 weeks after Ad-GFP administration, shows normal limb architecture. (B) A mutant limb from a 10 weeks old Gt(ROSA)26Sortm1(Smo/YFP)Amc/J mouse, 8 weeks after Ad-Cre administration with profound HO (arrowhead) forming in soft tissues.

### Example 9

### Treatment with ATO In Vivo Reduces HO Formation

Inhibitors of Hh signaling were tested for an ability to inhibit the formation of HO in vivo. Matings were established between Prx1-cre+; Gαₛ^{flox/-} male and Gαₛ^{flox/flox} female mice. Pregnant females at gestational days E13.5, E15.5 and E17.5 were injected intraperitoneally with 5 mg/kg ATO. Pregnant mice are first weighed and then injected with care so as to avoid injection into uteri. Mice were sacrificed at E18.5, pups were collected, and stained for bone formation by Alizarin Red-Alcian Blue staining. Prx1-cre+; Gαₛ^{flox/-} pups from mice injected with vehicle control produced HO as evidenced by enhanced red staining in soft tissues. Prx1-cre+; Gαₛ^{flox/-} pups from mice injected with 5 mg/kg ATO showed a significant reduction in HO in both forelimb and hind limb (FIG. 10). These results indicate that ATO and other inhibitors of hedgehog signaling are useful in preventing the formation of HO.

### Example 10

### Treatment with Hh Antagonists In Vivo Reduces Vascular Calcification

The ability of hedgehog pathway antagonists to block the formation of vascular calcification/ossification will be tested in established models.

Mating between either Prx1-cre^{+/-}; Gαₛ^{+/-} or Dermo1-cre^{+/-}; Gαₛ^{+/-} males and Gαₛflox/flox females will be made. The mutant mice from these matings with the genotypes of either Prx1-cre^{+/-}; Gαₛ^{flox/-} or Dermo1-cre^{+/-}; Gαₛ^{flox/-} show clear signs of vascular calcification and pathologic ossification at embryonic day 18.5 (E18.5) (FIGS. 1, 2 and 3). Pregnant females will be injected daily or every other day starting around E12.5 E13.5 with intraperitoneal injections of Hedgehog pathway antagonists (including ATO, GANT58, GANT61) and doses will range from 0.01 mg/kg to 100 mg/kg. Embryos will be collected at E18.5.

Mutant embryos with the genotypes Prx1-cre^{+/-}; Gαₛ^{flox/-} or Dermo1-cre^{+/-}; Gαₛ^{flox/-} who have come from females treated with Hh antagonists will be compared with mutant mice from female mice that have receive control injections. Using this approach, positive results demonstrated that ATO prevents the formation of pathologic ossification (FIG. 10).

Results showing GANT58 prevents to formation of pathologic ossification have also been obtained. The effects of Hh antagonists will be quantified in several ways:
1) Alizarin red-Alcian blue staining will be performed (as in FIG. 10) and morphometric analysis will permit quantification of the amount of red staining (bone) in the affected tissues;
2) RNA will be isolated from limbs and analyzed by qRT-PCR for markers of Hh signaling and bone formation [assumption is Hh antagonists will decrease expression of Hh signaling markers (Ptch1, Gli1, HIP) and decreased markers of bone formation (osterix, alkaline phosphatase, bone sialoprotein)];
3) affected tissue will be removed, fixed, and sectioned for histologic analysis [bone formation (e.g. subcutaneous or perivascular Von Kossa staining as in FIGS. 2A and 3A, respectively)) will be quantified in sections and compare mutants isolated from treated and untreated females]. For vascular calcification Alizarin red-Alcian blue staining will also be performed on the heart and great vessels of Dermo1-cre^{+/-}; Gαₛ^{flox/-} mutant mice. FIG. 3B shows that these mutant mice develop vascular ossification around the great vessels at E18.5. The heart and great vessels of Dermo1-cre^{+/-}; Gαₛ^{flox/-} mutant mice from females treated with Hh antagonists will be compared to Dermo1-cre^{+/-}; Gαₛ^{flox/-} mutant mice from females treated with vehicle controls. Morphometric analysis will allow quantification of ossification (Alizarin red staining) between treatment groups.

A second model of heterotopic ossification is described in O'Connor, 1998, Clin Orthopaedics Related Res 345:71-80, and in Shimono et al., 2011, Nature Medicine 17:454-60, hereby incorporated in their entirety. Briefly, 1 μg of human recombinant bone morphogenic protein 2 (BMP2) will be absorbed onto a small collagen disc and that disc will be inserted intramuscularly into an adult mouse. Within 5-6 days the released BMP2 will recruit bone-forming cells and within 2-3 weeks these cells will begin to ossify the tissue around the disc. The ability of treatment with Hh antagonists to block the formation of pathologic ossification will be measured. Mice will be treated, starting at day 1, with intraperitoneal injections given daily to every-other-day for 3 weeks. The doses will range from 0.01-100 mg/kg. At 3 weeks post disc insertion, the mice will be sacrificed and bone formation will be quantified using several strategies. Computed tomography (CT) and microcomputed tomography (μCT) will visualization and quantification of the size of the ossicle. Also, removing the ossicle and histologic sectioning will allow morphometric analysis and quantify bone formation (e.g. surface area, osteoblast number). Also, qRT-PCR for markers of bone formation (e.g. osterix, alkaline phosphatase, bone sialoprotein) will allow expression of genes associated with bone formation to be quantified. Controls will be mice who received injections of vehicle.

The ability of Hh pathway inhibitors to block the progression of pathologic ossification will be measured using this same BMP2 collagen disc implantation model. For this experiment, the BMP2/collagen disc will be implanted and the mice will be allowed to recover prior to injection of Hh pathway antagonists. Mice will be treated, starting at day 14, with intraperitoneal injections given daily to every-other-day for 2 weeks. The doses will range from 0.01-100 mg/kg. At 4 weeks post disc insertion, the mice will be sacrificed and bone formation will be quantified using several strategies. CT and μCT will be used to visualize and quantify the size of the ossicle. Also, removing the ossicle and histologic sectioning will allow morphometric analysis and quantification of bone formation (e.g. surface area, osteoblast number). Also, qRT-PCR for markers of bone formation (e.g. osterix, alkaline phosphatase, bone sialoprotein) will allow quantification of the expression of genes associated with bone formation. Controls will be mice who received injections of vehicle.

Dosage will be optimized for all experiments by identifying the highest concentration of the Hh pathway antagonist that is tolerated by the recipient (i.e. does not lead to death, does not induce abortion) and pathologic bone formation will be quantified. This dose will be established for both daily and every-other-day injections. Once this dose is identified the dose will be reduced by halves to identify lower doses of the compounds which are still effective at inhibiting pathologic bone formation.

For in vitro cellular models, bone marrow mesenchymal cells (BMMCs) will be isolated from adult Gαₛ^{flox/flox} mice and embryonic limb mesenchymal cells from E12.5 Gαₛ^{flox/flox} mice. These mice will be cultured and treated with either a cre recombinase-expressing or green fluorescent protein (GFP)-expressing adenovirus. The cre recombinase-expressing adenovirus will remove Gαₛ from the cells and these will function as the mutant ossifying cells. The GFP-expressing adenovirus will function as the control cells. Upon reaching confluence cells will be switched to osteogenic media [DMEM, 10% lot-selected FBS, 100 U/mL penicillin, 100 μg/mL streptomycin, 2 mM glutamine, 10-4M L-ascorbic acid 2-phosphate and 10 mM β-glycerol phosphate]. At all stages of culture cells will be treated either with media or media plus Hh inhibitor. The efficiency of Hh pathway inhibition by qRT-PCR for Ptch1, Gli1 and HIP will be quantified. Oosteoblast differentiation by qRT-PCR for specific markers [osterix (Osx), oollagen 1a1 (Col1a1), alkaline phosphatase (Alk Phos), bone sialoprotein (BSP), osteocalcin (OC)] will also be quantified. Von Kossa and Alizarin red staining will be used to measure in vitro bone formation. The time points to assay are: preconfluence and day 0, 2, 4, 7, 14, 21 in osteogenic media. As shown above, mutant BMMC express Hh signaling and show osteoblast differentiation under osteogenic conditions (FIG. 7).

To optimize dosage for these in vitro experiments will identify the highest concentration of the Hh pathway antagonist that allows normal cells growth (i.e. non-toxic) and block in vitro bone formation. Once this dose is identified, doses will be reduced by halves to identify lower doses of the compounds still effective at inhibiting Hh pathway inhibition and in vitro bone formation.

### Example 11

### Hedgehog Antagonists for Treating Non-Genetic HO

The purpose of this study is to determine if hedgehog antagonists are an effective treatment for patients with non-genetic forms of heterotopic ossification (HO, e.g., neurogenic injury, surgery, trauma or severe burns). The hedgehog antagonists may include one or more of the following drugs: vismodegib (GDC-0449, Genentech), bevacizumab, gemcitabine, nab-paclitaxel, FOLFIR, FOLFOX, RO4929097, cixutumumab, cisplatin, etoposide, LDAC, decitabine, daunorubicin, cytarabin, rosiglitazone, goserelin, leuprolide, capecitabine, fluorouracil, leucovorin, oxaliplatin, irinotecan, diclofenac, BMS-833923 (XL139), IPI-926—Infinity Pharmaceuticals, Inc., LDE225, LEQ506—Novartis Pharmaceuticals, TAK-441 Millennium Pharmaceuticals, Inc., or PF-04449913—Pfizer, alone or in combination therapy.

Condition: non-genetic forms of heterotopic ossification (HO)

Intervention: Drug: hedgehog antagonists through intraperitoneal injection (IP injection)

The clinical trial will be carried out, in accordance with applicable rules and regulations, e.g., 21 CFR 312 and 45 CFR 46.

Study Type: Interventional

Study Design: Allocation: Randomized

Endpoint Classification: Safety/Efficacy Study

Intervention Model: Single Group Assignment

Masking: Single Blind (Caregiver)

Primary Purpose: Treatment

Title: Hedgehog inhibitors for treatment of non-genetic forms of heterotopic ossification

Primary Outcome Measures:

Determine dose-response, tolerability, and adverse effects of Hh inhibitors in 100 patients with non-genetic forms of HO.

Estimated Enrollment: 100

Experimental Design:

In Phase I, a small group of healthy volunteers (20-100) will be used to evaluate the safety of the drug, determine a safe dosage range, and identify side effects of these compounds.

In Phase II, a larger group of patients (100-300) will be tested to determine the drug's effectiveness and further investigate its safety in a randomized manner relative to a placebo. This trial will last from two months to two years. Likely trial outcomes may include the reduced HO as measured by X-ray or computed tomography, quantification of blood markers of bone formation/turnover, analysis of joint stiffness/mobility, and quality of life assessment.

During Phase III, the drug will be given to large groups of patients (1,000-3,000) to confirm its effectiveness, monitor side effects, compare it to commonly used treatments, and collect information that will allow the experimental drug or treatment to be used safely. Patients will be randomized and blinded. This trial will likely last between one and four years. Likely trial outcomes may include the reduced sizes of HO as measured by X-ray or computed tomography, quantification of blood markers of bone formation/turnover, analysis of joint stiffness/mobility, and quality of life assessment.

In Phase IV, post marketing studies will gather additional information including the drug's risks, benefits, and optimal use.

### DETAILED DESCRIPTION

Sleep difficulties in children with autism spectrum disorders (ASD) are common reasons why parents seek medical intervention for their children. Identifying a safe and effective pharmacologic agent that promotes sleep in ASD would favorably impact the lives of these children and their families. In this study we plan to determine the dose-response, tolerability and any adverse effects of supplemental melatonin in 30 children with ASD. The melatonin dose levels are 1 mg, 3 mg, 6 mg, and 9 mg. After a 3 week baseline period, the child will begin melatonin at 1 mg and will dose escalate every three weeks until he/she is falling asleep within 30 minutes of bedtime at least 5/7 nights per week. No child will take more than 9 mg of supplemental melatonin.

Eligibility

Ages Eligible for Study: 10 Years to 60 Years

Genders Eligible for Study: Both

Accepts Healthy Volunteers: Yes

Criteria

Inclusion Criteria:
Patients with HO ages 10-60 years.
Diagnosis of HO based on X-ray or computed tomography, quantification of blood markers of bone formation/turnover, analysis of joint stiffness/mobility, and quality of life assessment.
Patients may take seasonal allergy medications.

Exclusion Criteria:
Patients taking medications other than those in the inclusion criteria.
Patients with other skeletal disorders.
Patients with liver disease or high fat diets, as Hh inhibitors may be affected in these children.
Patients with known genetic syndromes of skeletal defects.
Patients who have outside normal limits on blood work for complete blood count, liver and renal function and hormone levels of ACTH, cortisol, LH, FSH, prolactin, testosterone and estradiol.

1. A method of preventing or treating heterotopic ossification comprising administering a drug, wherein the drug is an antagonist of the Hedgehog pathway.

2. The method of claim 1, wherein the antagonist is selected from the group consisting of zerumbone epoxide, staurosporinone, 6-hydroxystaurosporinone, arcyriaflavin C, 5,6-dihyroxyarcyriaflavin A, physalin F, physalin B, cyclopamine, HPI-1, HPI-2, HPI-3, and HPI-4, vismodegib (GDC-0449, Genentech), bevacizumab, gemcitabine, nab-paclitaxel, FOLFIR, FOLFOX, R04929097, cixutumumab, cisplatin, etoposide, LDAC, decitabine, daunorubicin, cytarabin, rosiglitazone, goserelin, leuprolide, capecitabine, fluorouracil, leucovorin, oxaliplatin, irinotecan, diclofenac, BMS-833923 (XL139), IPI-926—Infinity Pharmaceuticals, Inc., LDE225, LEQ506—Novartis Pharmaceuticals, TAK-441 Millennium Pharmaceuticals, Inc., PF-04449913—Pfizer, arsenic trioxide, sodium arsenite, phenylarsine, GANT-58, GANT-61, and zerumbone, alone or in combination therapy.

3-5. (canceled)

6. The method of claim 2, wherein the antagonist comprises is arsenic trioxide (ATO).

7-8. (canceled)

9. The method of claim 1, wherein the antagonist targets pluripotent mesenchymal cells.

10. The method of claim 9, wherein the antagonist prevents proliferation or differentiation of mesenchymal cells.

11. The method of claim 10, wherein the antagonist alters expression of a gene expressed in the mesenchymal cells.

12. The method of claim 11, wherein the gene encodes a component of the Hedgehog pathway.

13-17. (canceled)

18. A method of inhibiting formation of heterotopic ossification comprising administering an antagonist of the Hedgehog pathway.

19. The method of claim 18, wherein the antagonist is administered to a human patient that is susceptible to heterotopic ossification.

20-21. (canceled)

22. The method of claim 19, wherein the patient experienced trauma.

23. The method of claim 22, wherein the patient experienced spinal cord injury, trauma and brain injuries, burns, fractures, muscle contusion, joint arthroplasty, lower motor neuron disorders, or hereditary disorders.

24. The method of claim 22, wherein the trauma is combat-related.

25-28. (canceled)

29. A method of treating heterotopic ossification (HO), vascular calcification, or pathologic mineralization using an antagonist of the Hedgehog pathway, comprising administering said antagonist to a subject in need thereof.

30. The method of claim 29, wherein the antagonist is selected from the group consisting of zerumbone epoxide, staurosporinone, 6-hydroxystaurosporinone, arcyriaflavin C, 5,6-dihyroxyarcyriaflavin A, physalin F, physalin B, cyclopamine, HPI-1, HPI-2, HPI-3, and HPI-4, arsenic trioxide, sodium arsenite, phenylarsine, GANT-58, GANT-61, and zerumbone.

31. The method of claim 30, wherein the antagonist comprises arsenic trioxide (ATO).

32. (canceled)

33. A method of using an antagonist of the Hedgehog pathway, comprising exposing mesenchymal cells to said antagonist to prevent activation of said cells.

34. The method of claim 33, wherein the antagonist prevents proliferation or differentiation of mesenchymal cells.

35-40. (canceled)


SEQUENCE LISTING (22 sequences)

SEQ ID NO: 1 - DNA, 22 bp, Artificial Sequence
  source: /note=“Description of Artificial Sequence Synthetic primer”
cacagcttctttgcagctcctt

SEQ ID NO: 2 - DNA, 19 bp, Artificial Sequence
  source: /note=“Description of Artificial Sequence Synthetic primer”
cgtcatccatggcgaactg

SEQ ID NO: 3 - DNA, 21 bp, Artificial Sequence
  source: /note=“Description of Artificial Sequence Synthetic primer”
caacgtcaagacggccgtgtg

SEQ ID NO: 4 - DNA, 21 bp, Artificial Sequence
  source: /note=“Description of Artificial Sequence Synthetic primer”
gacagaggcaaactgagcacc

SEQ ID NO: 5 - DNA, 20 bp, Artificial Sequence
  source: /note=“Description of Artificial Sequence Synthetic primer”
gcagaaggacaagcaggtct

SEQ ID NO: 6 - DNA, 20 bp, Artificial Sequence
  source: /note=“Description of Artificial Sequence Synthetic primer”
ccctctccgttaaacccatt

SEQ ID NO: 7 - DNA, 21 bp, Artificial Sequence
  source: /note=“Description of Artificial Sequence Synthetic primer”
ctctggagcagatttccaagg

SEQ ID NO: 8 - DNA, 20 bp, Artificial Sequence
  source: /note=“Description of Artificial Sequence Synthetic primer”
tgccgcagttcttttgaatg

SEQ ID NO: 9 - DNA, 22 bp, Artificial Sequence
  source: /note=“Description of Artificial Sequence Synthetic primer”
gaaagtcctattcacgccttga

SEQ ID NO: 10 - DNA, 25 bp, Artificial Sequence
  source: /note=“Description of Artificial Sequence Synthetic primer”
caaccttcttgctcacacatgtaag

SEQ ID NO: 11 - DNA, 20 bp, Artificial Sequence
  source: /note=“Description of Artificial Sequence Synthetic primer”
gggaaaaacaggtcatcagc

SEQ ID NO: 12 - DNA, 18 bp, Artificial Sequence
  source: /note=“Description of Artificial Sequence Synthetic primer”
atccaccaaccaaagggc

SEQ ID NO: 13 - DNA, 24 bp, Artificial Sequence
  source: /note=“Description of Artificial Sequence Synthetic primer”
cccactggctcctcggttctctcc

SEQ ID NO: 14 - DNA, 25 bp, Artificial Sequence
  source: /note=“Description of Artificial Sequence Synthetic primer”
gctbgaaaggtcagcgtatggcttc

SEQ ID NO: 15 - DNA, 20 bp, Artificial Sequence
  source: /note=“Description of Artificial Sequence Synthetic primer”
caccctcaagagcctgagtc

SEQ ID NO: 16 - DNA, 20 bp, Artificial Sequence
  source: /note=“Description of Artificial Sequence Synthetic primer”
gttcgggctgatgtaccagt

SEQ ID NO: 17 - DNA, 23 bp, Artificial Sequence
  source: /note=“Description of Artificial Sequence Synthetic primer”
cacgcgatgcaacaccactcagg

SEQ ID NO: 18 - DNA, 21 bp, Artificial Sequence
  source: /note=“Description of Artificial Sequence Synthetic primer”
gcatgtccccgggctcaaaga

SEQ ID NO: 19 - DNA, 24 bp, Artificial Sequence
  source: /note=“Description of Artificial Sequence Synthetic primer”
taccggccacgctactttctttat

SEQ ID NO: 20 - DNA, 22 bp, Artificial Sequence
  source: /note=“Description of Artificial Sequence Synthetic primer”
gaccgccagctcgttttcatcc

SEQ ID NO: 21 - DNA, 22 bp, Artificial Sequence
  source: /note=“Description of Artificial Sequence Synthetic primer”
accctggctgcgctctgtctct

SEQ ID NO: 22 - DNA, 23 bp, Artificial Sequence
  source: /note=“Description of Artificial Sequence Synthetic primer”
gatgcgtttgtaggcggtcttca